(19) United States
(12) Patent Application Publication
(10) Pub. No.: US 2016/0376661 A1
(43) Pub. Date: Dec. 29, 2016

(54) A METHOD FOR PREDICTING RESPONSIVENESS TO A TREATMENT WITH AN EGFR INHIBITOR

(71) Applicant: INTEGRAGEN, Evry (FR)

(72) Inventor: Raphaele THIEBAUT, Versailles (FR)

(73) Assignee: Integragen, Evry (FR)

(21) Appl. No.: 15/038,826

(22) PCT Filed: Nov. 26, 2014
(86) PCT No.: PCT/EP2014/075651

§ 371 Date: May 24, 2016

(30) Foreign Application Priority Data
Nov. 26, 2013 (EP) 13306619.1

(51) Int. Cl. C12Q 1/68

(52) U.S. Cl. CPC C12Q 1/6886; C12Q 2600/178; C12Q 2600/106; C12Q 2600/158

(57) ABSTRACT

The present invention relates to a method for predicting whether a patient with a cancer is likely to respond to an epidermal growth factor receptor (EGFR) inhibitor, which method comprises determining the expression level of at least one target gene of hsa-miR-31-3p (SEQ ID NO:1) miRNA in a sample of said patient, wherein said target gene of hsa-miR-31-3p is selected from DBNDD2 and EPB41 L4B. The invention also relates to kits for measuring the expression of DBNDD2 and/or EPB41 L4B and at least one other parameter positively or negatively correlated to response to EGFR inhibitors. The invention also relates to therapeutic uses of an EGFR inhibitor in a patient predicted to respond to said EGFR inhibitor.

### TECHNICAL FIELD OF THE INVENTION

The present invention provides methods for individualizing chemotherapy for cancer treatment, and particularly for evaluating a patient's responsiveness to one or more epidermal growth factor receptor (EGFR) inhibitors prior to treatment with such agents, based on the determination of the expression level of at least one target gene of hsa-miR-31-3p (SEQ ID NO:1) miRNA, wherein said target gene of hsa-miR-31-3p is selected from DBNDD2 and EPB41L4B.

### BACKGROUND OF THE INVENTION

The epidermal growth factor receptor (EGFR) pathway is crucial in the development and progression of human epithelial cancers. The combined treatment with EGFR inhibitors has a synergistic growth inhibitory and pro-apoptotic activity in different human cancer cells which possess a functional EGFR-dependent autocrine growth pathway through to a more efficient and sustained inhibition of Akt.

EGFR inhibitors have been approved or tested for treatment of a variety of cancers, including non-small cell lung cancer (NSCLC), head and neck cancer, colorectal carcinoma, and Her2-positive breast cancer, and are increasingly being added to standard therapy. EGFR inhibitors, which may target either the intracellular tyrosine kinase domain or the extracellular domain of the EGFR target, are generally plagued by low population response rates, leading to ineffective or non-optimal chemotherapy in many instances, as well as unnecessary drug toxicity and expense. For example, a reported clinical response rate for treatment of colorectal carcinoma with cetuximab (a chimeric monoclonal antibody targeting the extracellular domain of EGFR) is about 11% (Cunningham et al, N Engl Med 2004; 351: 337-45), and a reported clinical response rate for treatment of NSCLC with erlotinib is about 8.9% (Shepherd F A, et al, N Engl J Med 2005; 353:123-132).

In particular resistance has been observed in case of KRAS mutation.

In colorectal cancer, as KRAS mutations are clearly associated with resistance to anti-EGFR antibodies (Lievre et al, Cancer Res. 2006 66(8):3992-5), one of the major challenges is to identify, in non-mutated KRAS patients, other markers that can predict lack of response to this therapy. Among them, amplification or activating mutations of oncogenes and inactivating mutations of tumor suppressor genes described above are relevant candidates, such as the level of activation of EGFR downstream signaling pathway evaluated by the measurement of EGFR downstream phosphoprotein expression.

In lung cancer, three groups of patients are emerging: one counts the patients with EGFR mutated tumors for which the use of EGFR tyrosine kinase inhibitors (EGFR TKI) was proven to improve outcome, the second counts the patients with KRAS mutated tumors for which anti-EGFR therapies are probably not the good alternatives, and the third group counts the non-EGFR and non-KRAS mutated tumors for which response cannot be predicted. No marker linked to drug response in the non-mutated tumor group has proved valuable so far.

Thus, there is a need for predicting patient responsiveness to EGFR inhibitors prior to treatment with such agents, so as to better individualize patent therapy.

There are many documents in the prior art concerning the involvement of micro RNAs (miRNAs) in sensitivity or resistance to various anticancer treatments. In particular, PCT/EP2012/073535 describes an in vitro method for predicting whether a patient with a cancer is likely to respond to an epidermal growth factor receptor (EGFR)inhibitor, which comprises determining the expression level of hsa-miR-31-3p (previously named hsa-miR-31*, SEQ ID NO:1) miRNA in a sample of said patient. More particularly, the lower the expression of hsa-miR-31-3p is, the more likely the patient is to respond to the EGFR inhibitor treatment.

Similarly, there are many documents in the prior art concerning the involvement of various genes in sensitivity or resistance to various anticancer treatments. However, in most cases, studies are partial, incomplete, and actually do not permit a true prediction of clinical response or non-response to treatment. Indeed, in many cases, studies are limited to the analysis of the expression of genes in vitro, in cell lines sensitive or resistant to a particular treatment, or in tumor cells isolated from a patient tumor. In addition, in many studies, while differences in expression level between two populations of cells or patients are shown, no threshold value or score actually permitting to predict response or non-response in a new patient are provided. This is partly linked to the first shortage that many studies lack data obtained in a clinical setting. Moreover, even when some data obtained in a clinical setting is presented, these data are most of the time only retrospective, and data validating a prediction method in an independent cohort are often lacking.

In view of various shortcomings of prior art studies, there is still a need for true and validated methods for predicting response to EGFR inhibitors in patients for which such therapy is one of several options. The present invention provides a response to this need.

DBNDD2 (dysbindin (dystrobrevin binding protein 1) domain containing 2) has been disclosed to be a binding partner of human casein kinase-1 (Yin H et al. Biochemistry. 2006 Apr. 25; 45(16):5297-308). In addition, using microarray global profiling, it has been found, among many other genes, to be differentially expressed in various tumor cells (WO2010065940; WO2010059742; WO2009131710; WO2007112097), or between cancer cells sensitive or resistant torapamycin (WO2011017106) or tamoxifen (WO2010127338). However, this gene does not seem to have been specifically associated to cancer, and no involvement of this gene in prediction of response to EGFR inhibitors has been disclosed.

EPB41L4B (erythrocyte membrane protein band 4.1 like 4B) is a protein of the FERM family proteins, which can link transmembrane proteins to the cytoskeleton or link kinase and/or phosphatase enzymatic activity to the plasma membrane, and have been described to be involved in carcinogenesis and metastasis. In particular, EPB41 L4B (also known as EHM2) has been associated to increased aggressiveness of prostate cancer (Wang J, et al. Prostate. 2006 Nov. 1; 66(15):1641-52; Schulz W A, et al. BMC Cancer. 2010 Sep. 22; 10:505) and breast cancer (Yu H et al. Mol Cancer Res 2010; 8:1501-1512). This gene has thus been associated to aggressiveness and poor prognosis of at least two types of cancer. Moreover, it has been found to be differentially expressed between cancer cell lines sensitive and resistant to taxotere (docetaxel, see WO2007072225 and WO2008138578). However, there has been no disclosure of its association to the ability of a cancer patient to respond or not to EGFR inhibitors.

The inventors implemented a new database incorporating information from the 6 databases, which may be interrogated either based on the name of a miRNA, or based on a gene name. In the first case (query based on miRNA name), the database returns genes names considered as candidate targets of the queried miRNA, based on published or structural information, candidate target genes being ranked from the most probable to the less probable based on available information. When the query is based on a gene name, the database returns candidates miRNAs, for which the queried gene might (or not) be a target.

### SUMMARY OF THE INVENTION

With the aim to understand why increased expression of hsa-miR-31-3p is associated to lower response to EGFR inhibitor treatment, the inventors tried to identify target genes of this miRNA. For this purpose, they transfected three colorectal adenocarcinoma (CRC) cell lines that naturally weakly express hsa-miR-31-3p with a mimic of hsa-miR-31-3p or a negative control mimic and analyzed genes differentially expressed between cell lines overexpressing or expressing weakly hsa-miR-31-3p. A total of 74 genes significantly down- or up-regulated was identified. Since miRNAs function mainly by decreasing expression of their target genes, the inventors focused on the 47 down-regulated genes. To limit the number of candidate targets and avoid the false direct target genes, the inventors further performed in silico analyses based on information available in 6 databases relating to miRNAs and candidate targets. It is important to note that, most miRNA target genes provided in public databases are not validated, but only more or less probable candidates, based on structural or fragmental experimental data. 25 candidate target genes of hsa-miR-31-3p were selected for further analysis on this basis. The inventors further analyzed the expression of these candidate target genes of hsa-miR-31-3p in tumor samples of patients treated with EGFR inhibitors, whose treatment response status based on RECIST criteria were known.

Based on these analyses, the inventors surprisingly found that DBNDD2 and EPB41L4B are both hsa-miR-31-3p target genes, since their expression is significantly down-regulated by overexpression of hsa-miR-31-3p in cancer cell lines, and that each of these genes is independently significantly associated to the ability of cancer patients to respond to EGFR inhibitor treatment. They further confirmed that each of these genes may alone be used for reliably predicting response to EGFR inhibitors in cancer patients. None of the other 23 candidate target genes of hsa-miR-31-3p was found to be significantly associated to the ability of cancer patients to respond to EGFR inhibitor treatment, although some of these genes were considered in databases as a candidate target gene of hsa-miR-31-3p with higher probability, such as HAUS4, and known to be associated to cancer, such as STAT3, FEM1A, EHBP1 and SEC31A. This clearly indicates that mere association of a gene to cancer is not sufficient to reasonably expect that the gene may be used as a biomarker of response to a particular cancer treatment. This also illustrates that only a few of the numerous candidate target genes of a particular miRNA disclosed in public databases are true targets of this miRNA, and that the true targets are not necessarily the best ranked candidates.

Surprisingly, the two genes found to be significantly down-regulated in patients not responding to EGFR inhibitor treatment are a gene not specifically known to be associated to cancer (DBNDD2) and a gene known to be associated to cancer (EPB41L4B), but for which high expression level was associated to poor prognosis. In contrast, in the present invention, it is a low expression of EPB41L4B that is associated to absence of response to EGFR inhibitors, and thus to poor prognosis. These results further confirm that biomarkers of prognosis (in general) may not be reasonably expected to be also biomarkers of response to a particular treatment.

Based on the results obtained by the inventors (see Example 1), the present invention provides an in vitro method for predicting whether a patient with a cancer is likely to respond to an epidermal growth factor receptor (EGFR) inhibitor, which comprises determining the expression level of at least one target gene of hsa-miR-31-3p (SEQ ID NO:1) miRNA in a sample of said patient, wherein said target gene of hsa-miR-31-3p is selected from DBNDD2 and EPB41L4B.

Preferably the patient has a KRAS wild-type cancer.

The cancer preferably is a colorectal cancer, preferably a metastatic colorectal cancer. In a most preferred embodiment, the invention provides an in vitro method for predicting whether a patient with a metastatic colorectal carcinoma is likely to respond to an epidermal growth factor receptor (EGFR) inhibitor, such as cetuximab or panitumumab, which method comprises determining the expression level of at least one target gene of hsa-miR-31-3p (SEQ ID NO:1) miRNA in a tumor sample of said patient, wherein said target gene of hsa-miR-31-3p is selected from DBNDD2 and EPB41L4B.

The invention also provides a kit for determining whether a patient with a cancer is likely to respond to an epidermal growth factor receptor (EGFR) inhibitor, comprising or consisting of: reagents for determining the expression level of at least one target gene of hsa-miR-31-3p (SEQ ID NO:1) miRNA in a sample of said patient, wherein said target gene of hsa-miR-31-3p is selected from DBNDD2 and EPB41L4B, and reagents for determining at least one other parameter positively or negatively correlated to response to EGFR inhibitors.

The invention further relates to an EGFR inhibitor for use in treating a patient affected with a cancer, wherein the patient has been classified as being likely to respond, by the method according to the invention.

The invention also relates to the use of an EGFR inhibitor for the preparation of a drug intended for use in the treatment of cancer in patients that have been classified as “responder” by the method of the invention.

The invention also relates to a method for treating a patient affected with a cancer, which method comprises (i) determining whether the patient is likely to respond to an EGFR inhibitor, by the method of the invention, and (ii) administering an EGFR inhibitor to said patient if the patient has been determined to be likely to respond to the EGFR inhibitor.

### BRIEF DESCRIPTION OF THE FIGURES

FIG. 1: Correlation between log₂ expression levels of DBNDD2 (in FIG. 1A) and EPB41L4B (in FIG. 1B) and hsa-miR-31-3p in the 20 mCRC patients of Example 1.

FIG. 2: Correlation between log₂ expression levels of DBNDD2 and hsa-miR-31-3p in the 20 mCRC patients of Example 2.

FIG. 3: In A: Nomogram tool established based on log₂ expression of DBNDD2 in the 20 mCRC patients of Example 2, in order to predict risk of progression (i.e. risk of non-response) of mCRC patients treated with anti-EGFR-based chemotherapy.

FIG. 4: Multivariate Cox proportional hazards models with DBNDD2 expression as covariate in the 20 mCRC patients of Example 2.

FIG. 5: Correlation between log₂ expression levels of DBNDD2 (in FIG. 5A) and EPB41L4B (in FIG. 5B) and hsa-miR-31-3p in the 42 mCRC patients of Example 3.

FIG. 6: Expression of DBNDD2 (in FIG. 6A) and EPB41L4B (in FIG. 6B) in patients of Example 3 according to their risk of progression (low or high), as predicted based on hsa-miR-31-3p expression level.

### DETAILED DESCRIPTION OF THE INVENTION

### Definitions

The “patient” may be any mammal, preferably a human being, whatever its age or sex. The patient is afflicted with a cancer. The patient may be already subjected to a treatment, by any chemotherapeutic agent, or may be untreated yet.

The cancer is preferably a cancer in which the signaling pathway through EGFR is involved. In particular, it may be e.g. colorectal, lung, breast, ovarian, endometrial, thyroid, nasopharynx, prostate, head and neck, kidney, pancreas, bladder, or brain cancer (Ciardello F et al. N Engl J Med. 2008 Mar. 13; 358(11):1160-74; Wheeler D L et al. Nat Rev Clin Oncol. 2010 September; 7(9): 493-507; Zeineldin R et al. J Oncol. 2010; 2010:414676; Albitar L et al. Mol Cancer 2010; 9:166; Leslie K K et al. Gynecol Oncol. 2012 November; 127(2):345-50; Mimeault M et al. PLoS One. 2012; 7(2):e31919; Liebner D A et al. Ther Adv Endocrinol Metab. 2011 October; 2(5):173-95; Leboulleux S et al. Lancet Oncol. 2012 September; 13(9):897-905; Pan J et al. Head Neck. 2012 Sep. 13; Chan S L et al. Expert Opin Ther Targets. 2012 March; 16 Suppl 1:S63-8; Chu H et al. Mutagenesis. 2012 Oct. 15; Li Y et al. Oncol Rep. 2010 October; 24(4):1019-28; Thomasson M et al. Br J Cancer 2003, 89:1285-1289; Thomasson M et al. BMC Res Notes. 2012 May 3; 5:216). In certain embodiments, the tumor is a solid tissue tumor and/or is epithelial in nature. For example, the patient may be a colorectal carcinoma patient, a Her2-positive or Her2-negative (in particular triple negative, i.e. Her2-negative, estrogen receptor negative and progesterone receptor negative) breast cancer patient, a non-small cell lung cancer (NSCLC) patient, a head and neck cancer patient (in particular a squamous-cell carcinoma of the head and neck patient), a pancreatic cancer patient, or an endometrial cancer patient. More particularly, the patient may be a colorectal carcinoma patient, a Her2-positive or Her2-negative (in particular triple negative) breast cancer patient, a lung cancer (in particular a NSCLC) patient, a head and neck cancer patient (in particular a squamous-cell carcinoma of the head and neck patient), or a pancreatic cancer patient.

In a preferred embodiment, the cancer is a colorectal cancer, still preferably the cancer is a metastatic colorectal cancer. Indeed, data presented in Example 1 clearly indicate that DBNDD2 or EPB41L4B expression level may be used as a predictor of response to EGFR inhibitors (and in particular to anti-EGFR monoclonal antibodies such as cetuximab and panitumumab) treatment in colorectal cancer.

These results, obtained in a cancer in which the EGFR signaling pathway is known to be involved, clearly suggest that DBNDD2 and/or EPB41L4B expression level might be used as a predictor of response to EGFR inhibitors (and in particular to anti-EGFR monoclonal antibodies such as cetuximab and panitumumab) in any other cancer in which the EGFR signaling pathway is known to be involved, such as lung, ovarian, endometrial, thyroid, nasopharynx, prostate, head and neck, kidney, pancreas, bladder, or brain cancer. Therefore, in another preferred embodiment, the cancer is a Her2-positive or Her2-negative (in particular triple negative) breast cancer, preferably a Her2-negative (in particular triple negative) breast cancer.

In still another preferred embodiment, the cancer is a lung cancer, in particular a non-small cell lung cancer (NSCLC).

In still another preferred embodiment, the cancer is a pancreatic cancer.

Since the prediction relates to EGFR inhibitors treatment, the patient's tumor is preferably EGFR positive.

Preferably, the patient has a KRAS wild-type tumor, i.e., the KRAS gene in the tumor of the patient is not mutated in codon 12, 13 (exon 1), or 61 (exon 3). In other words, the KRAS gene is wild-type on codons 12, 13 and 61.

Wild type, i.e. non mutated, codons 12, 13 (exon 1), and 61 (exon 3) respectively correspond to glycine (Gly, codon 12), glycine (Gly, codon 13), and glutamine (Gln, codon 61). The wild-type reference KRAS amino acid sequence may be found in Genbank accession number NP_004976.2 (SEQ ID NO:24).

Especially the KRAS gene of the patient's tumor does not show any of the following mutations (Bos. Cancer Res 1989; 49:4682-4689; Edkins et al. Cancer Biol Ther. 2006 August; 5(8): 928-932; Demiralay et al. Surgical Science, 2012, 3, 111-115):

### Gly12Ser (GGT>AGT)

### Gly12Arg (GGT>CGT)

### Gly12Cys (GGT>TGT)

### Gly12Asp (GGT>GAT)

### Gly12Ala (GGT>GCT)

### Gly12Val (GGT>GTT)

### Gly13Arg (GGC>CGC)

### Gly13Cys (GGC>TGC)

### Gly13Asp (GGC>GAC)

### Gly13Ala (GGC>GCC)

### Gly13Val (GGC>GTC)

Preferably, the KRAS gene of the patient's tumor does also not show any of the following mutations (Demiralay et al. Surgical Science, 2012, 3, 111-115):

### Gly12Phe (GGT>TTT)

### Gly13Ser (GGC>AGC)

Preferably, the KRAS gene of the patient's tumor does also not show any of the following mutations (Bos. Cancer Res 1989; 49:4682-4689; Tam et al. Clin Cancer Res 2006; 12:1647-1653; Edkins et al. Cancer BiolTher. 2006 August; 5(8): 928-932; Demiralay et al. Surgical Science, 2012, 3, 111-115):

### Gln61His (CAA>CAC)

### Gln61His (CAA>CAT)

### Gln61Arg (CAA>CGA)

### Gln61Leu (CAA>CTA)

### Gln61Glu (CAA>GAA)

### Gln61Lys (CAA>AAA)

### Gln61 Pro (CAA>CCA)

Any method known in the art may be used to know the KRAS status of the patient.

For example, a tumor tissue is microdissected and DNA extracted from paraffin-embedded tissue blocks. Regions covering codons 12, 13, and 61 of the KRAS gene are amplified using polymerase chain reaction (PCR). Mutation status is determined by allelic discrimination using PCR probes (Laurent-Puig P, et al, J Clin Oncol. 2009, 27(35):5924-30) or by any other methods such as pyrosequencing (Ogino S, et al. J Mol Diagn 2008; 7:413-21).

The “sample” may be any biological sample derived from a patient, which contains nucleic acids. Examples of such samples include fluids (including blood, plasma, saliva, urine, seminal fluid), tissues, cell samples, organs, biopsies, etc. Preferably the sample is a tumor sample, preferably a tumor tissue biopsy or whole or part of a tumor surgical resection. The sample may be collected according to conventional techniques and used directly for diagnosis or stored. A tumor sample may be fresh, frozen or paraffin-embedded. Usually, available tumor samples are frozen or paraffin-embedded, most of the time paraffin-embedded. The inventors have shown that both frozen and paraffin-embedded tumor samples may be used.

By a “reference sample”, it is meant a tumor sample (notably a tumor biopsy or whole or part of a tumor surgical resection) of a patient whose positive or negative response to an EGFR inhibitor treatment is known. Preferably, a pool of reference samples comprises at least one (preferably several, more preferably at least 5, more preferably at least 6, at least 7, at least 8, at least 9, at least 10) responder patient(s) and at least one (preferably several, more preferably at least 6, at least 7, at least 8, at least 9, at least 10) non responder patient(s). The highest the number of responders (also referred to as “positive”) and non-responders (also referred to as “negative”) reference samples, the better for the reliability of the method of prediction according to the invention.

Within the context of this invention, a patient who is “likely to respond” or is “responder” refers to a patient who may respond to a treatment with an EGFR inhibitor, i.e. at least one of his symptoms is expected to be alleviated, or the development of the disease is stopped, or slowed down. Complete responders, partial responders, or stable patients according to the RECIST criteria (Eisenhauer et al, European Journal of Cancer, 2009, 45:228-247) are considered as “likely to respond” or “responder” in the context of the present invention.

In solid tumors, the RECIST criteria are an international standard based on the presence of at east one measurable lesion. “Complete response” means disappearance of all target lesions; “partial response” means 30% decrease in the sum of the longest diameter of target lesions, “progressive disease” means 20% increase in the sum of the longest diameter of target lesions, “stable disease” means changes that do not meet above criteria.

More preferably, a “responder” patient is predicted to show a good progression free survival (PFS), i.e. the patient is likely to survive at least 25 weeks without aggravation of the symptoms of the disease, and/or such patient shows a good overall survival (OS), i.e. the patient is likely to survive at least 14 months.

The term “predicting” or “prognosis” refers to a probability or likelihood for a patient to respond to the treatment with an EGFR inhibitor.

According to the invention, the sensitivity of tumor cell growth to inhibition by an EGFR inhibitor is predicted by whether and to which level such tumor cells express hsa-miR-31-3p target genes DBNDD2 and EPB41L4B.

The term “treating” or “treatment” means stabilizing, alleviating, curing, or reducing the progression of the cancer.

A “miRNA” or “microRNA” is a single-stranded molecule of about 21-24 nucleotides, preferably 21-23 in length, encoded by genes that are transcribed from DNA but not translated into protein (non-coding RNA); instead they are processed from primary transcripts known as pri-miRNA to short stem-loop structures called pre-miRNA and finally to functional miRNA. During maturation, each pre-miRNA gives rise to two distinct fragments with high complementarity, one originating from the 5′ arm the other originating from the 3′ arm of the gene encoding the pri-miRNA. Mature miRNA molecules are partially complementary to one or more messenger RNA (mRNA) molecules, and their main function is to downregulate gene expression.

There is an international nomenclature of miRNAs (see Ambros V et al, RNA 2003 9(3):277-279; Griffiths-Jones S. NAR 2004 32(Database Issue):D109-D111; Griffiths-Jones S et al. NAR 2006 34(Database Issue):D140-D144; Griffiths-Jones S et al. NAR 2008 36(Database Issue):D154-D158; and Kozomara A et al. NAR 2011 39(Database Issue):D152-D157), which is available from miRBase at http://www.mirbase.org/. Each miRNA is assigned a unique name with a predefined format, as follows:
For a mature miRNA: sss-miR-X-Y, wherein “
   sss is a three letters code indicating the species of the miRNA, “hsa” standing for human,
   the upper case “R” in miR indicates that it is referred to a mature miRNA. However, some authors in the literature abusively use “mir” also for mature miRNA. In this case, it may be recognized that it is referred to a mature miRNA by the presence of “-Y”,
   X is the unique arbitrary number assigned to the sequence of the miRNA in the particular species, which may be followed by a letter if several highly homologous miRNAs are known. For instance, “20a” and “20b” refer to highly homologous miRNAs.
   Y indicates whether the mature miRNA, which has been obtained by cutting of the pre-miRNA, corresponds to the 5′ arm (Y is then “5p”) or 3′ arm (Y is then “3p”) of the gene encoding the pri-mRNA. In previous international nomenclature of miRNAs, “-Y” was not present. The two mature miRNAs obtained either from the 5′ or the 3′ arm of the gene encoding the pri-miRNA were then distinguished by the presence or absence of a “*” sign just after n. The presence of the “*” sign indicated that the sequence corresponded to the less often detected miRNA. Since such classification was subject to changes, a new nomenclature using the “3p” and “5p” code has been implemented.
For a pri-miRNA:sss-mir-X, wherein
   sss is a three letters code indicating the species of the miRNA, “hsa” standing for human,
   the lower case “r” in mir indicates that it is referred to a pri-miRNA and not to a mature miRNA, which is confirmed by the absence of “-Y”,
   n is the unique arbitrary number assigned to the sequence of the miRNA in the particular species, which may be followed by a letter if several highly homologous miRNAs are known.

Each miRNA is also assigned an accession number for its sequence.

The miRNA targeted by the two genes detected in the present invention (DBNDD2 and EPB41L4B) is hsa-miR-31-3p (previously named hsa-miR-31*). In this name, “hsa” means that it relates to a human miRNA, “miR” refers to a mature miRNA, “31” refers to the arbitrary number assigned to this particular miRNA, and “3p” means that the mature miRNAs has been obtained from the 3′ arm of the gene encoding the pri-miRNA.

hsa-miR-31-3p is
(SEQ ID NO: 1)
UGCUAUGCCAACAUAUUGCCAU
(Accession number MIMAT0004504 on
http://www.mirbase.org)

“DBNDD2” is the official symbol of NCBI Entrez Gene database for “dysbindin (dystrobrevin binding protein 1) domain containing 2” gene (official name and symbol approved by the HUGO Gene Nomenclature Committee (HGNC)), located in humans in chromosome 20 (20q13.12). It corresponds to UniGene database accession number Hs.730643. Further symbols used for this gene include CK1BP (for “casein kinase-1 binding protein”), HSMNP1, RP3-453C12.9, and C20orf35. It is also known as “SCF apoptosis response protein 1”. Five isoforms (a to e) of this protein are known, encoded by several mRNA variants, as detailed in Table 1 below.

TABLE 1
isoforms of DBNDD2 and corresponding mRNA and protein reference sequences
provided by NCBI EntrezGene database, on Jul. 1, 2013.

| DBNDD2 isoform | mRNA RefSeq | Protein RefSEq |
|---|---|---|
| Isoform a | NM_001048221.2 (SEQ ID NO: 2) | NP_001041686.1 (SEQ ID NO: 11) |
| | NM_001048223.2 (SEQ ID NO: 3) | NP_001041688.1 (SEQ ID NO: 12) |
| | NM_001197139.1 (SEQ ID NO: 4) | NP_001184068.1 (SEQ ID NO: 13) |
| | NM_001197140.1 (SEQ ID NO: 5) | NP_001184069.1 (SEQ ID NO: 14) |
| Isoform b | NM_001048222.2 (SEQ ID NO: 6) | NP_001041687.1 (SEQ ID NO: 15) |
| | NM_001048224.2 (SEQ ID NO: 7) | NP_001041689.1 (SEQ ID NO: 16) |
| Isoform c | NM_001048225.2 (SEQ ID NO: 8) | NP_001041690.2 (SEQ ID NO: 17) |
| Isoform d | NM_001048226.2 (SEQ ID NO: 9) | NP_001041691.2 (SEQ ID NO: 18) |
| Isoform e | NM_018478.3 (SEQ ID NO: 10) | NP_060948.3 (SEQ ID NO: 19) |

“EPB41L4B” is the official symbol of NCBI Entrez Gene database for “erythrocyte membrane protein band 4.1 like 4B” gene (official name and symbol approved by the HGNC), located in humans in chromosome 9 (9q31-q32). It corresponds to UniGene database accession number Hs.591901. Further symbols used for this gene include CG1 and EHM2 (for “Expressed in Highly Metastatic cells 2”). It is also known as “FERM-containing protein CG1”. Two isoforms (1 and 2) of this protein are known, encoded by two mRNA variants, as detailed in Table 2 below.

TABLE 2
isoforms of EPB41L4Band corresponding mRNA and protein
reference sequences provided by NCBI EntrezGene database,
as updated on Jul. 1, 2013.

| EPB41L4B | | |
|---|---|---|
| isoform | mRNA RefSeq | Protein RefSEq |
| Isoform 1 | NM_018424.2 (SEQ ID NO: 20) | NP_060894.2 |
| | | (SEQ ID NO: 22) |
| Isoform 2 | NM_019114.3 (SEQ ID NO: 21) | NP_061987.3 |
| | | (SEQ ID NO: 23) |

Methods of Detecting DBNDD2 and/or EPB41L4B Expression Levels in a Sample

The expression level of hsa-miR-31-3p target gene(s) DBNDD2 and/or EPB41L4B may be determined by any technology known by a person skilled in the art. In particular, each gene expression level may be measured in vitro, starting from the patient's sample, at the genomic and/or nucleic acid and/or proteic level. In a preferred embodiment, the expression profile is determined by measuring in vitro the amount of nucleic acid transcripts of each gene. In another embodiment, the expression profile is determined by measuring in vitro the amount of protein produced by each of the genes.

Such measures are made in vitro, starting from a patient's sample, in particular a tumor sample, and necessary involve transformation of the sample. Indeed, no measure of a specific gene expression level can be made without some type of transformation of the sample.

Most technologies rely on the use of reagents specifically binding to the gene DNA, transcripts or proteins, thus resulting in a modified sample further including the detection reagent.

In addition, most technologies also involve some preliminary extraction of DNA, mRNA or proteins from the patient's sample before binding to a specific reagent. The claimed method may thus also comprise a preliminary step of extracting DNA, mRNA or proteins from the patient's sample. In addition, when mRNAs are extracted, they are generally retrotranscribed into cDNA, which is more stable than mRNA. The claimed methods may thus also comprise a step of retrotranscribing mRNA extracted from the patient's sample into cDNA.

Detection by mass spectrometry does not necessary involve preliminary binding to specific reagents. However, it is most of the time performed on extracted DNA, mRNA or proteins. Even when preformed directly on the sample, without preliminary extraction steps, it involves some extraction of molecules from the sample by the laser beam, which extracted molecules are then analysed by the spectrometer.

In any case, no matter which technology is used, the state of the sample after measure of a gene expression level has been transformed compared to the initial sample taken from the patient.

The amount of nucleic acid transcripts can be measured by any technology known by a person skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a person skilled in the art, including nucleic microarrays, quantitative PCR, next generation sequencing and hybridization with a labelled probe.

In particular, real time quantitative RT-PCR (qRT-PCR) may be useful. In some embodiments, qRT-PCR can be used for both the detection and quantification of RNA targets (Bustin et al., 2005, Clin. Sci., 109:365-379). Quantitative results obtained by qRT-PCR can sometimes be more informative than qualitative data, and can simplify assay standardization and quality management. Thus, in some embodiments, qRT-PCR-based assays can be useful to measure hsa-miR-31-3p target gene(s) DBNDD2 and/or EPB41L4B expression levels during cell-based assays. The qRT-PCR method may be also useful in monitoring patient therapy. qRT-PCR is a well-known and easily available technology for those skilled in the art and does not need a precise description. Examples of qRT-PCR-based methods can be found, for example, in U.S. Pat. No. 7,101,663. Commercially available qRT-PCR based methods (e.g., Taqman® Array) may for instance be employed, the design of primers and/or probe being easily made based on the sequences of DBNDD2 and/or EPB41L4B disclosed in Tables 1 and 2 above.

Nucleic acid assays or arrays can also be used to assess in vitro the expression level of the gene in a sample, by measuring in vitro the amount of gene transcripts in a patient's sample. In some embodiments, a nucleic acid microarray can be prepared or purchased. An array typically contains a solid support and at least one nucleic acid (cDNA or oligonucleotide) contacting the support, where the oligonucleotide corresponds to at least a portion of a gene. Any suitable assay platform can be used to determine the presence of hsa-miR-31-3p target gene(s) DBNDD2 and/or EPB41L4B in a sample. For example, an assay may be in the form of a membrane, a chip, a disk, a test strip, a filter, a microsphere, a multiwell plate, and the like. An assay system may have a solid support on which a nucleic acid (cDNA or oligonucleotide) corresponding to the gene is attached. The solid support may comprise, for example, a plastic, silicon, a metal, a resin, or a glass. The assay components can be prepared and packaged together as a kit for detecting a gene. To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the person skilled in the art.

In another embodiment, the measure in vitro of hsa-miR-31-3p target gene(s) DBNDD2 and/or EPB41L4B expression level(s) may be performed by sequencing of transcripts (mRNA or cDNA) of the gene extracted from the patient's sample.

In still another embodiment, the measure in vitro of hsa-miR-31-3p target gene(s) DBNDD2 and/or EPB41L4B expression level(s) may be performed by the use of a protein microarray, for measuring the amount of the gene encoded protein in total proteins extracted from the patient's sample.

### Classifying the Patient

Classification Based on DBNDD2 and/or EPB41L4B Expression Level(s)

The higher the expression of hsa-miR-31-3p target gene(s) DBNDD2 and/or EPB41L4B is, the better for the patient. Therefore, the higher the level of expression of hsa-miR-31-3p target gene(s) DBNDD2 and/or EPB41L4B is, the more likely the patient is to respond to the EGFR inhibitor treatment. In an embodiment, the patient is considered as “responder”, or likely to respond to a treatment with an EGFR inhibitor, when the expression of hsa-miR-31-3p target gene(s) DBNDD2 and/or EPB41L4B is higher than a control value.

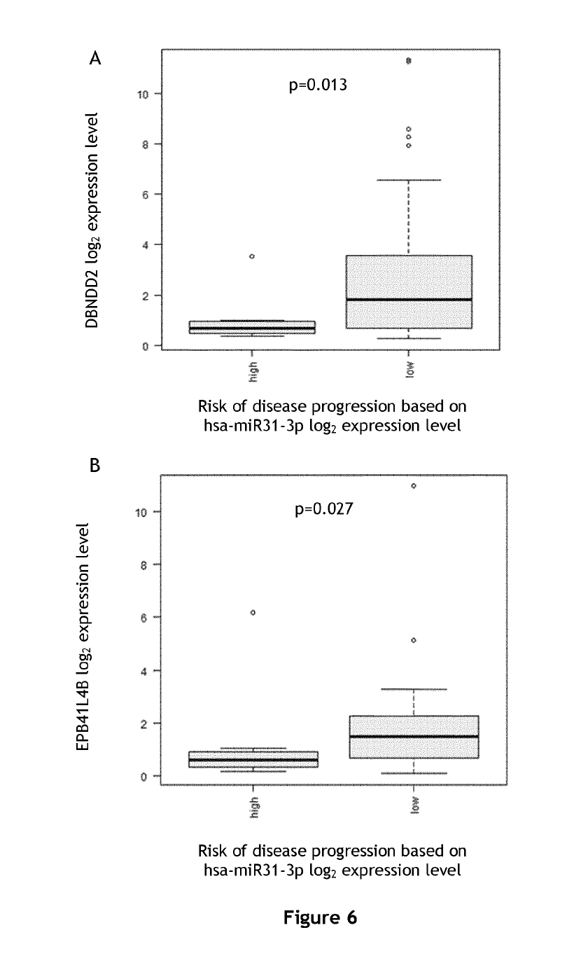

Such a control value may be determined based on a pool of reference samples, as defined above. In particular, FIG. 6 clearly shows that, based on a pool of reference samples, a control value for DBNDD2 and EPB41L4B level of expression (the logged DBNDD2:EPB41L4B level of expression) may be defined that permits to predict response or non-response to EGFR inhibitor treatment.

However, in a preferred embodiment, the method further comprises determining a prognostic score or index based on the expression level of at least one of hsa-miR-31-3p target gene(s) DBNDD2 and EPB41L4B, wherein the prognostic score indicates whether the patient is likely to respond to the EGFR inhibitor. In particular, said prognosis score may indicate whether the patient is likely to respond to the EGFR inhibitor depending if it is higher or lower than a predetermined threshold value (dichotomized result). In another embodiment, a discrete probability of response or non-response to the EGFR inhibitor may be derived from the prognosis score.

The probability that a patient responds to an EGFR inhibitor treatment is linked to the probability that this patient survives, with or without disease progression, if the EGFR inhibitor treatment is administered to said patient.

As a result, a prognosis score may be determined based on the analysis of the correlation between the expression level of at least one of hsa-miR-31-3p target gene(s) DBNDD2 and EPB41L4B and progression free survival (PFS) or overall survival (OS) of a pool of reference samples, as defined above. A PFS and/or OS score, which is a function correlating PFS or OS to the expression level of at least one of hsa-miR-31-3p target gene(s) DBNDD2 and EPB41L4B, may thus be used as prognosis score for prediction of response to an EGFR inhibitor. Preferably, a PFS score is used, since absence of disease progression is a clear indicator of response to the EGFR inhibitor treatment.

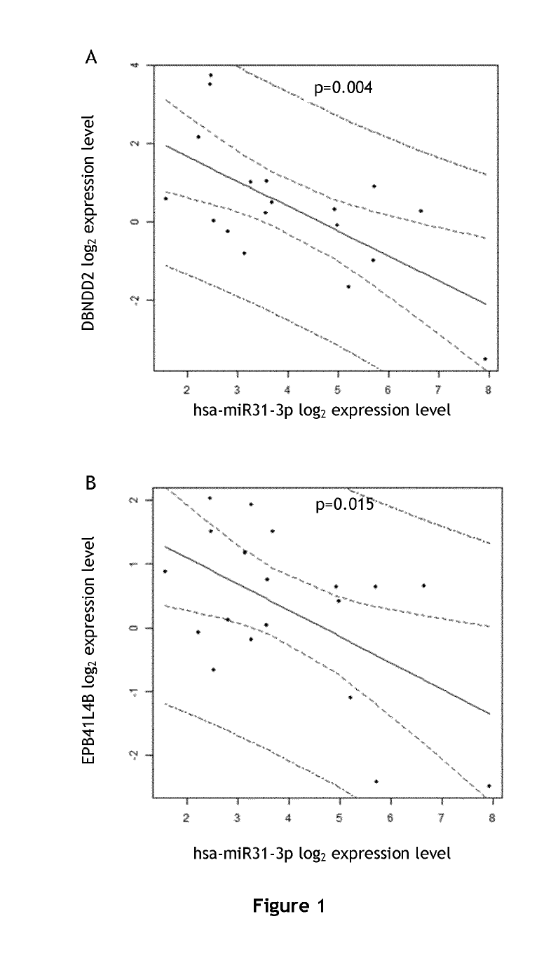

Experimental data obtained by the inventors shows that the probability for a patient to respond to an EGFR inhibitor treatment is linearly and negatively correlated to the logged expression level of each of DBNDD2 and EPB41L4B (see FIGS. 1, 2 and 5). In a preferred embodiment, said prognosis score is thus represented by the following formula:

Prognosis score=a*x+b,

wherein x is the logged expression level of DBNDD2 (preferably log in base 2, referred to as “log₂”) and/or EPB41L4B measured in the patient's sample, and a and b are parameters that have been previously determined based on a pool of reference samples, as defined above.

Depending if a is positive/negative, the patient may then be predicted as responding to the EGFR inhibitor if his/her prognosis score is greater than or equal to/lower than or equal to a threshold value c, and not responding to the EGFR inhibitor if his/her prognosis score is lower than/greater than threshold value c, wherein the value of c has also been determined based on the same pool of reference samples:
If a is positive, the patient may then be predicted as responding to the EGFR inhibitor if his/her prognosis score is greater than or equal to threshold value c, and not responding to the EGFR inhibitor if his/her prognosis score is lower than threshold value c.
Alternatively, if a is negative, then the patient may be predicted as responding to the EGFR inhibitor if his/her prognosis score is lower than or equal to threshold value c, and not responding to the EGFR inhibitor if his/her prognosis score is greater than threshold value c.

In another embodiment, a discrete probability of response or non-response to the EGFR inhibitor may be derived from the above a*x+b prognosis score. A precise correlation between the prognosis score and the probability of response to the EGFR inhibitor treatment may be determined based on the same set of reference samples. Depending if a is positive/negative, a higher/lower prognosis score indicates a higher probability of response to the EGFR inhibitor treatment:
If a is positive, the higher the prognosis score, the higher is the probability of response to the EGFR inhibitor treatment (i.e. the lower is the probability of disease progression in the case of a PFS score).
Alternatively, if a is negative, then the lower the prognosis score, the higher is the probability of response to the EGFR inhibitor treatment (i.e. the lower is the probability of disease progression in the case of a PFS score).

This prediction of whether a patient with a cancer is likely to respond to an EGFR inhibitor may also be made using a nomogram. In a nomogram, points scales are established for each variable of a score of interest. For a given patient, points are allocated to each of the variables by selecting the corresponding points from the points scale of each variable. For a discrete variable (such as a gene expression level), the number of points attributed to a variable is linearly correlated to the value of the variable. For a dichotomized variable (only two values possible), two distinct values are attributed to each of the two possible values or the variable. The score of interest is then calculated by adding the points allocated for each variable (total points). Based on the value of the score, the patient may then be given either a good or bad response prognosis depending on whether the composite score is inferior or superior to a threshold value (dichotomized score), or a probability of response or non-response to the treatment.

It is clear that nomograms are mainly useful when several distinct variables are combined in a composite score (see below the possibility to use composite scores combining DBNDD2 and EPB41L4B expression levels; DBNDD2 and/or EPB41L4B expression levels and hsa-miR-31-3p expression level; or DBNDD2 and/or EPB41L4B expression level(s) and BRAF status). However, a nomogram may also be used to represent a prognosis score based on only one variable, such as DBNDD2 or EPB41L4B expression level. In this case, total points correspond to points allocated to the single variable.

An example of a nomogram permitting determination of a risk of progression (i.e. of a risk of non-response to EGFR inhibitors) in colorectal cancer patients based on DBNDD2 logged (log₂) expression level is displayed in FIG. 3 (see also Example 2 below).

Therefore, in an embodiment of the method for predicting whether a patient with a cancer is likely to respond to an EGFR inhibitor according to the invention, the method further comprises determining a risk of non-response based on a nomogram calibrated based on a pool of reference samples. The nomogram may be calibrated based on OS or PFS data. If calibrated based on OS, the risk of non-response corresponds to a risk of death. If calibrated based on PFS, the risk of non-response corresponds to a risk of disease progression (see FIG. 3).

As explained above, each of DBNDD2 and EPB41L4B has been found to be a target gene of hsa-miR-31-3p and to be independently significantly associated to response to EGFR inhibitors, so that the expression level of only one of DBNDD2 and EPB41L4B may be measured and used for prediction in a method according to the invention.

However, the method according to the invention may also comprise determining the expression levels of both DBNDD2 and EPB41L4B in the patient's sample, and predicting response or non-response based on the combined expression of DBNDD2 and EPB41L4B. A composite score combining the expression levels of DBNDD2 and EPB41L4B may notably be created based on a pool of reference samples. A nomogram may also be used to combine the expression levels of DBNDD2 and EPB41L4B and obtain the composite score, which may then be correlated to the risk of non-response (i.e. the risk of disease progression for a PFS score).

Classification Based on DBNDD2 and/or EPB41L4B Expression Level(s) and Further Parameters Positively or Negatively Correlated to Response to EGFR Inhibitors

While response to EGFR inhibitors can be predicted based only on the expression level of at least one of hsa-miR-31-3p target genes DBNDD2 and EPB41L4B (see Examples 1, 2 and 3), the method according to the invention may also comprise determining at least one other parameter positively or negatively correlated to response to EGFR inhibitors.

In this case, a composite score combining the expression level(s) of DBNDD2 and/or EPB41L4B and the other parameter(s) may notably be created based on a pool of reference samples.

A nomogram, in which points scales are established for each variable of the composite score, may also be used to combine the expression level(s) of DBNDD2 and/or EPB41L4B and the other parameter(s), and obtain the composite score, which may then be correlated to the risk of non-response (i.e. the risk of disease progression for a PFS score). For a given patient, points are allocated to each of the variables by selecting the corresponding points from the points scale of each variable. For a discrete variable (such as DBNDD2 or EPB41L4B expression level or age), the number of points attributed to a variable is linearly correlated to the value of the variable. For a dichotomized variable (only two values possible, such as BRAF mutation status or gender), two distinct values are attributed to each of the two possible values or the variable.

A composite score is then calculated by adding the points allocated for each variable (total points). Based on the value of the composite score, the patient may then be given either a good or bad response prognosis depending on whether the composite score is inferior or superior to a threshold value (dichotomized score), or a probability of response or non-response to the treatment.

The points scale of each variable, as well the threshold value over/under which the response prognosis is good or bad or the correlation between the composite score and the probability of response or non-response may be determined based on the same pool of reference samples.

Such other parameters positively or negatively correlated to response to EGFR inhibitors may notably be selected from:
age;
gender;
the expression level of hsa-miR-31-3p, which may be measured at the genomic and/or nucleic (in particular by measuring the amount of nucleic acid transcripts of each gene) and/or proteic level, by any method disclosed above for measuring the expression level of DBNDD2 and EPB41L4B; and/or
the presence or absence of at least one mutation positively or negatively correlated to response to EGFR inhibitors.
Such mutations may be detected by any method known to those skilled in the art and notably include those mentioned in Table 3 below

| | | | Genbank reference | |
|---|---|---|---|---|
| Gene | Unigene | | wild-type protein | |
| symbol | number | Chromosome | sequence(s) | Mutation* |
| Kras | Hs.505033 | 12 | NP_004976.2 | G12 |
| | | | (SEQ ID NO: 24) | G13 |
| | | | | Q61 |
| | | | | K117N |
| | | | | A146 |
| BRAF | Hs.550061 | 7 | NP_004324.2 | V600 |
| | | | (SEQ ID NO: 25) | |
| NRAS | Hs.486502 | 1 | NP_002515.1 | G12 |
| | | | (SEQ ID NO: 26) | G13 |
| | | | | Q61 |
| | | | | K117 |
| | | | | A146T |
| PIK3CA | Hs.553498 | 3 | NP_006209.2 | E545 |
| | | | (SEQ ID NO: 27) | H1047 |
| EGFR | Hs.488293 | 7 | NP_005219.2 | S492R |
| | | | (SEQ ID NO: 28); | |
| | | | NP_958441.1 | |
| | | | (SEQ ID NO: 29); | |
| | | | NP_958439.1 | |
| | | | (SEQ ID NO: 30); | |
| AKT1 | Hs.525622 | 14 | NP_001014431.1 | E17K |
| | | | (SEQ ID NO: 31); | |
| | | | NP_001014432.1 | |
| | | | (SEQ ID NO: 32); | |
| | | | NP_005154.2 | |
| | | | (SEQ ID NO: 33) | |

* Mutations are defined by mention of the codon number in the protein, preceded by the one letter code for the wild-type amino acid, and optionally followed by the replacement amino acid. When no replacement amino acid is mentioned, the replacement amino acid may be any amino acid different from the wild-type amino acid.

### EGFR Inhibitors

The present invention makes it possible to predict a patient's responsiveness to one or more epidermal growth factor receptor (EGFR) inhibitors prior to treatment with such agents.

The EGRF inhibitor may be an EGFR tyrosine kinase inhibitor, or may alternatively target the extracellular domain of the EGFR target. In certain embodiments, the EGFR inhibitor is a tyrosine kinase inhibitor such as Erlotinib, Gefitinib, or Lapatinib, or a molecule that targets the EGFR extracellular domain such as Cetuximab or Panitumumab.

Preferably the EGFR inhibitor is an anti-EGFR antibody, preferably a monoclonal antibody, in particular Cetuximab or Panitumumab.

Molecules that target the EGFR extracellular domain, including anti-EGFR monoclonal antibodies such as Cetuximab or Panitumumab, are mainly used in the treatment of colorectal cancer or breast cancer treatment. As a result, if the patient's cancer is colorectal cancer (in particular metastatic colorectal cancer) or breast cancer, then the method according to the invention may preferably be used to predict response to molecules that target the EGFR extracellular domain, and in particular to anti-EGFR monoclonal antibodies, such as Cetuximab or Panitumumab.

Conversely, tyrosine kinase EGFR inhibitors are mainly used in the treatment of lung cancer (in particular non-small cell lung cancer, NSCLC), so that if the patient's cancer is lung cancer (in particular non-small cell lung cancer, NSCLC), then the method according to the invention may preferably be used to predict response to tyrosine kinase EGFR inhibitors, such as Erlotinib, Gefitinib, or Lapatinib.

In pancreatic cancer or head and neck cancer (in particular squamous cell carcinoma of the head and neck (SCCHN)), both tyrosine kinase EGFR inhibitors and anti-EGFR monoclonal antibodies are being tested as therapy, so that if the patient's cancer is pancreatic cancer or head and neck cancer (in particular squamous cell carcinoma of the head and neck (SCCHN)), then the method according to the invention may be used to predict response either to tyrosine kinase EGFR inhibitors (such as Erlotinib, Gefitinib, or Lapatinib) or to anti-EGFR monoclonal antibodies (such as Cetuximab or Panitumumab).

Cetuximab and Panitumumab are currently the clinically mostly used anti-EGFR monoclonal antibodies. However, further anti-EGFR monoclonal antibodies are in development, such as Nimotuzumab (TheraCIM-h-R3), Matuzumab (EMD 72000), and Zalutumumab (HuMax-EGFr). The method according to the invention may also be used to predict response to these anti-EGFR monoclonal antibodies or any other anti-EGFR monoclonal antibodies (including fragments) that might be further developed, in particular if the patient is suffering from colorectal cancer (in particular metastatic colorectal cancer), breast cancer, pancreatic cancer or head and neck cancer (in particular squamous cell carcinoma of the head and neck (SCCHN)).

Similarly, Erlotinib, Gefitinib, and Lapatinib are currently the clinically mostly used tyrosine kinase EGFR inhibitors. However, further tyrosine kinase EGFR inhibitors are in development, such as Canertinib (CI-1033), Neratinib (HKI-272), Afatinib (BIBW2992), Dacomitinib (PF299804, PF-00299804), TAK-285, AST-1306, ARRY334543, AG-1478 (Tyrphostin AG-1478), AV-412, OSI-420 (DesmethylErlotinib), AZD8931, AEE788 (NVP-AEE788), Pelitinib (EKB-569), CUDC-101, AG 490, PD153035 HCl, XL647, and BMS-599626 (AC480). The method according to the invention may also be used to predict response to these tyrosine kinase EGFR inhibitors or any other tyrosine kinase EGFR inhibitors that might be further developed, in particular if the patient is suffering from of lung cancer (in particular non-small cell lung cancer, NSCLC), pancreatic cancer, or head and neck cancer (in particular squamous cell carcinoma of the head and neck (SCCHN)).

### Kits

The present invention also relates to a kit for determining whether a patient with a cancer is likely to respond to an epidermal growth factor receptor (EGFR) inhibitor, comprising or consisting of:
a) reagents for determining the expression level of at least one target gene of hsa-miR-31-3p (SEQ ID NO:1) miRNA in a sample (preferably a tumor sample, such as a tumor biopsy or whole or part of a tumor surgical resection) of said patient, wherein said target gene of hsa-miR-31-3p is selected from DBNDD2 and EPB41L4B, and
b) reagents for determining at least one other parameter positively or negatively correlated to response to EGFR inhibitors.
   Such reagents may notably include reagents for:
      i) determining the expression level of at least one miRNA positively or negatively correlated to response to EGFR inhibitors, in particular hsa-miR-31-3p (SEQ ID NO:1) miRNA or particular hsa-miR-31-5p (SEQ ID NO:34) in a sample (preferably a tumor sample, such as a tumor biopsy or whole or part of a tumor surgical resection) of said patient, and/or,
      ii) detecting at least one mutation positively or negatively correlated to response to EGFR inhibitors, such as those mentioned in Table 3 above.

Reagents for determining the expression level of at least one of hsa-miR-31-3p target gene(s) DBNDD2 and EPB41L4B or of at least one miRNA positively or negatively correlated to response to EGFR inhibitors, in particular hsa-miR-31-3p itself or hsa-miR-31-5p, in a sample of said patient, may notably comprise or consist of primers pairs (forward and reverse primers) and/or probes specific for at least one of hsa-miR-31-3p target gene(s) DBNDD2 and EPB41L4B or a microarray comprising a sequence specific for at least one of hsa-miR-31-3p target gene(s) DBNDD2 and EPB41L4B. The design of primers and/or probe can be easily made by those skilled in the art based on the sequences of DBNDD2 and/or EPB41L4B disclosed in Tables 1 and 2 above.

Reagents for detecting at least one mutation positively or negatively correlated to response to EGFR inhibitors may include at least one primer pair for amplifying whole or part of the gene of interest before sequencing or a set of specific probes labeled with reporter dyes at their 5′ end, for use in an allelic discrimination assay, for instance on an ABI 7900HT Sequence Detection System (Applied Biosystems, Foster City, Calif.) (see Laurent-Puig P, et al, J Clin Oncol. 2009, 27(35):5924-30 and Lièvre et al. J Clin Oncol. 2008 Jan. 20; 26(3):374-9 for detection of BRAF mutation V600).

The kit of the invention may further comprise instructions for determining whether the patient is likely to respond to the EGFR inhibitor based on the expression level of at least one of hsa-miR-31-3p target gene(s) DBNDD2 and EPB41L4B and the other tested parameter. In particular, a nomogram including points scales of all variables included in the composite score and correlation between the composite score (total number of points) and the prediction (response/non-response or probability of response or non-response) may be included.

### Drugs, Therapeutic Uses and Methods of Treating

The method of the invention predicts patient responsiveness to EGFR inhibitors at rates that match reported clinical response rates for the EGFR inhibitors.

It is thus further provided a method for treating a patient with a cancer, which method comprises administering to the patient at least one EGFR inhibitor, wherein the patient has been predicted (or classified) as “responder” or “likely to respond” by the method as described above.

In particular, the invention concerns a method for treating a patient affected with a cancer, which method comprises (i) determining whether the patient is likely to respond to an EGFR inhibitor, by the method according to the invention, and (ii) administering an EGFR inhibitor to said patient if the patient has been determined to be likely to respond to the EGFR inhibitor.

The method may further comprise, if the patient has been determined to be unlikely to respond to the EGFR inhibitor a step (iii) of administering an alternative anticancer treatment to the patient. Such alternative anticancer treatment depends on the specific cancer and on previously tested treatments, but may notably be selected from radiotherapy, other chemotherapeutic molecules, or other biologics such as monoclonal antibodies directed to other antigens (anti-Her2, anti-VEGF, anti-EPCAM, anti-CTLA4 . . . ). In particular, in the case of colorectal cancer, if the patient has been determined to be unlikely to respond to the EGFR inhibitor, the alternative anticancer treatment administered in step (iii) may be selected from:
a VEGF inhibitor, in particular an anti-VEGF monoclonal antibodies (such as bevacizumab), advantageously in combination with FOLFOX (a combination of leucovorin (folinic acid), 5-fluorouracil (5-FU), and oxaliplatin) or FOLFIRI (a combination of leucovorin (folinic acid), 5-fluorouracil (5-FU), and irinotecan) chemotherapy.
Alternatively, if the patient has already been treated unsuccessfully with a VEGF inhibitor, optionally in combination with FOLFOX or FOLFIRI chemotherapy, it may be administered with 5-FU, optionally in combination with Mitomycin B. Best supportive care, defined as a treatment administered with the intent to maximize quality of life without a specific antineoplastic regimen (i.e. not an anticancer treatment) may further be administered to the patient.

Another subject of the invention is an EGFR inhibitor, for use in treating a patient affected with a cancer, wherein the patient has been classified as being likely to respond by the method as defined above. The invention also relates to an EGFR inhibitor for use in treating a patient affected with a cancer, wherein said treatment comprises a preliminary step of predicting if said patient is or not likely to respond to the EGFR inhibitor by the method as defined above, and said EGFR inhibitor is administered to the patient only is said patient has been predicted as likely to respond to the EGFR inhibitor by the method as defined above. Said patient may be affected with a colorectal cancer, more particularly a metastatic colorectal cancer. Alternatively, said patient may be affected with a breast cancer, in particular a triple negative breast cancer. Alternatively, said patient may be affected with a lung cancer, in particular a non-small cell lung cancer (NSCLC). Alternatively, said patient may be affected with a head and neck cancer, in particular a squamous-cell carcinoma of the head and neck. Alternatively, said patient may be affected with a pancreatic cancer. The invention also relates to the use of an EGFR inhibitor for the preparation of a medicament intended for use in the treatment of cancer in patients that have been classified as “responder” by the method of the invention as described above.

In a preferred embodiment the EGFR inhibitor is an anti-EGFR antibody, preferably cetuximab or panitumumab. Alternatively, the EGFR inhibitor may be a tyrosine kinase EGFR inhibitor, in particular Erlotinib, Gefitinib, or Lapatinib.

In preferred embodiments:
the patient is afflicted with a colorectal cancer, in particular a metastatic colorectal cancer, and the EGFR inhibitor is an anti-EGFR antibody, preferably cetuximab or panitumumab;
the patient is afflicted with a breast cancer, in particular a triple negative breast cancer, and the EGFR inhibitor is an anti-EGFR antibody, preferably cetuximab or panitumumab;
the patient is afflicted with a lung cancer, in particular a non-small cell lung cancer (NSCLC), and the EGFR inhibitor is a tyrosine kinase EGFR inhibitor, in particular Erlotinib, Gefitinib, or Lapatinib;
the patient is afflicted with a head and neck cancer, in particular a squamous-cell carcinoma of the head and neck, or a pancreatic cancer, and the EGFR inhibitor is an anti-EGFR antibody (preferably cetuximab or panitumumab) or a tyrosine kinase EGFR inhibitor (in particular Erlotinib, Gefitinib, or Lapatinib).

The examples and figures illustrate the invention without limiting its scope.

### EXAMPLES

### Example 1

### DBNDD2 and EPB41L4B are Targets of Hsa-miR-31-3p and Independently Predict Response to EGFR Inhibitors

### Patients and Methods

### Patients

The set of patients was made of 20 mCRC (metastatic colorectal cancer) patients, 14 males, 6 females. The median of age was 66.49±11.9 years. All patients received a combination of irinotecan and cetuximab. The number of chemotherapy lines before the introduction of Cetuximab was recorded. The median of follow-up until progression was 20 weeks and the median overall survival was 10 months. All tumor sample came from resections and were fixed in formalin and paraffin embedded (FFPE).

### Cell Culture and Transfection

We selected 3 colorectal adenocarcinoma cell lines from the American Type Culture Collection (ATCC, Manassas, Calif.) that express weakly hsa-miR-31-3p: HTB-37, CCL-222 and CCL-220-1. HTB-37 cells were maintained in a Dulbecco's Modified Eagle Medium (DMEM) culture medium with stable glutamine with 20% Fetal Bovine serum and 1% Penicillin/Streptomycin. CCL-222 and CCL-220-1 cells were maintained in a RPMI 1640 culture media with stable glutamine with 10% fetal bovine serum. The cells were incubated at a temperature of 37° C. with 5% CO2.

All the cells were transfected with miRVana miRNA mimic negative control or hsa-miR-31-3p miRVana miRNA mimic (Ambion). For CCL-222, transfections were done with 2 μl of lipofectamine RNAiMax with reverse transfection protocol according to the manufacturer's protocol using 25 pmol of MiRNA mimic and 60 000 cells in a 12 wells plate. For CCL-220-1 and HBT27, transfections were done using 4 μl of RiboCellin (BioCellChallenge, Toulon, France) according to the manufacturer's protocol using 12.5 pmol of miRNA mimic and 100 000 cells in a 12 wells plate. For all the cell lines, cells were harvested 24 h hours after transfection and Qiazol was used to protect RNA until extraction of total RNA with miRNeasy extraction kit (Qiagen). To assess for the efficacy of the transfection, specific quantification of miRNA hsa-miR-31-3p expression level was done as described below.

### Measurement of Gene Expression

Gene expression microarray was performed using the AffymetrixHuman Gene 1.0. Fifty ng of total RNA was reverse transcribed following the Ovation PicoSL WTA System V2 (Nugen, San Carlos, Calif.). Then, amplification was done based on SPIA technology. After purification according to Nugen protocol, 2.5 μg of single strand DNA was used for fragmentation and biotin labelling using Encore Biotin Module (Nugen). After control of fragmentation using Bioanalyzer 2100, cDNA was then hybridized to GeneChip® human Gene 1.0 ST (Affymetrix) at 45° C. for 17 hours. After hybridization, chips were washed on the fluidic station FS450 following specific protocols (Affymetrix) and scanned using the GCS3000 7G. The image was then analyzed with Expression Console software (Affymetrix) to obtain raw data (CELfiles) and metrics for Quality Controls.

qRT-PCR validation of the target expression on cell lines and FFPE patients samples were performed on 20 ng of total RNA for FFPE samples or 50 ng of total RNA cell culture samples using ABI7900HT Real-Time PCR System (Applied Biosystem). All reactions were performed in triplicate. Expression levels were normalized to the RNA18S and GAPDH levels through the ΔΔCt method.

### In Silico Analysis

We developed a data portal integrating up-to-date microRNA target predictions from six individual prediction databases (PITA, picTar 5-way, Targetscan, microRNA.org, MicroCosm and miRDB). This portal allows to determine microRNAs potentially co-targeted by a list of candidate genes, taking into account the number of microRNA prediction databases predicting each microRNA/target relationship and the rank of prediction of each miRNA from individual prediction databases. This database has been updated in November 2012 to perform the reported analysis.

### Statistical Analyses

Survival statistical analysis was performed using the R packages ‘survival’ and ‘rms’. Univariate and multivariate analyses used a Cox proportional regression hazard model and generated a hazard ratio (HR). Nomograms were developed based on Cox proportional regression hazard models, which predict the probability of free-progression survival.

False-discovery rate (FDR)-adjusted p-values were calculated using the Benjamini and Hochberg procedure for multiple testing correction. The cor.test function was used to calculate Pearson correlations between expression values together with matching p-values. Statistical significance was set at p<0.05 for all analyses.

### Results

Three CRC cell lines that weakly express hsa-miR-31-3p were transfected with hsa-miR-31-3p mimic or with a mimic control. The transfection efficacy was attested by an average rise of hsa-miR-31-3p level of 1500 times without mortality or growth defect. Expression profile analysis of the transfected cells allowed us to identify 47 genes significantly down-regulated (fc<0.77, p<0.05), and 27 genes significantly up-regulated by hsa-miR-31-3p (fc<1.3, p<0.05), as described in Table 4 below.

TABLE 4
List of the genes with a fc <0.77 or fc >1.3 and a pvalue ≧0.05
identified in the expression array made on the 3 cell lines (fc: fold
change in expression between cell lines transfected with hsa-miR-31-3p
mimic and cell lines transfected with a mimic control)

| | Gene ID |
|---|---|
| Down-regulated | AGPAT9; AMFR; B4GALT1; C12orf52; C2; C22orf13; CA12; |
| Genes | CD177; CSGALNACT2; DBNDD2; EHBP1; EPB41L4B; FAM108A1; |
| (47) | FEM1A; GMFB; GOLGA6L9; HAUS4; HLA-DRA; HSPB11; LCE2C; |
| | LPGAT1; LSM14B; LYN; NECAP1; OSGIN2; OSTM1; PCDHA6; |
| | PCP4; PLEKHB2; PNP; POLR2K; POTEM; RHPN2; SEC31A; |
| | SNORA70; STAT3; TCEB3CL; TMA7; TMEM171; TMEM8A; |
| | TMPRSS11E; TNFRSF1A; UBE2H; UGT2B7; VDAC1; WDR45L; |
| | XPNPEP3 |
| Up-regulated | ARL1; ARDDC4; ATMIN; BBX; CALU; CCND3; CEP170; CFB; |
| Genes(27) | ERCC5; FAM75A7; GINS3; LILRA6; MAP2K4; MBTPS1; MET; |
| | NKIRAS1; NRBF2; PIP4K2A; PTPMT1; RBPJ; SNX29P2; STMN1; |
| | SUSD1; TGIF1; TMEFF1; UNC119B; WSB1 |

As the role of a microRNA includes degradation of its transcript target, we studied if the database including information from 6 web-available predicts the 54 down-regulated genes as hsa-miR-31-3p putative target. The database may be queried either by miRNA name, or by gene name. When a miRNA name is queried, the database returns a list of candidate target genes, ranked by order of probability (from the most probable to the less probable) that the genes are true targets of the queried miRNA, based on structural and potential experimental data included in the database. Conversely, when a gene name is queried, the database returns a list of miRNA candidates, ranked by order of probability (from the most probable to the less probable) that the miRNAs truly target the queried gene, based on structural and potential experimental data included in the database. The database was queried with hsa-miR-31-3p name and with the names of genes found to be down-regulated in CRC cell lines overexpressing hsa-miR-31-3p (47 genes, cf Table 4).

Table 5 below shows down-regulated genes of Table 4, including DBNDD2 and EPB41L4B, which were identified as a putative direct target of has-miR-31-3p. It also indicates the rank of hsa-miR-31-3p if the database was queried using the gene name, and the rank of the gene if the database was queried using hsa-miR-31-3p name.

TABLE 5
Target predictions from in silico database are indicated for the
down-regulated genes depending on the request: Column 2:
database was interrogated with a gene of interest, and reported
all candidate microRNAs potentially targeting this gene, ranked
from the most likely to the less likely. The rank of hsa-miR-31-3p
and the total number of microRNA candidates are indicated;
Column 3: database was interrogated with hsa-miR-31-3p, and
reported all putative targets, ranked from the most likely to the
less likely for a total of 1620 putative targeted genes. Then rank
of the queried gene is indicated. Only down-regulated genes
listed in hsa-miR-31-3p 1620 putative targeted genes are presented
in Table 5. Data relating to DBNDD2 and EPB41L4B are in bold.

| | Hsa-miR-31-3p ranking by the | Gene ranking by |
|---|---|---|
| | gene/Number of predicted | hsa-miR-31-3p (on |
| Genes ID | microRNA | 1620 putative targets) |
| AMFR | 72/216 | 293 |
| B4GALT1 | 94/223 | 293 |
| CA12 | 48/182 | 293 |
| CSGALNACT2 | 89/242 | 293 |
| DBNDD2 | 41/139 | 293 |
| EHBP1 | 13/361 | 10 |
| EPB41L4B | 101/425 | 86 |
| FEM1A | 21/125 | 293 |
| GMFB | 211/348 | 293 |
| HAUS4 | 1/110 | 16 |
| HSPB11 | 37/279 | 101 |
| LSM14B | 52/288 | 101 |
| OSGIN2 | 119/289 | 293 |
| OSTM1 | 86/305 | 67 |
| PCP4 | 18/109 | 115 |
| PLEKHB2 | 93/257 | 293 |
| PNP | 9/216 | 31 |
| POLR2K | 5/162 | 2 |
| POTEM | 47/210 | 293 |
| SEC31A | 9/238 | 78 |
| STAT3 | 37/240 | 166 |
| UBE2H | 120/303 | 293 |
| VDAC1 | 29/213 | 173 |
| WDR45L | 39/154 | 293 |
| XPNPEP3 | 145/583 | 293 |

Among the 47 down-regulated genes, 25 were predicted to be putative direct target of hsa-miR-31-3p and displayed a good rank in the prediction database. This number and the ranking of the genes are significant (P<0.0001 for both test by permutation test). As expected, none but one of the 27 up-regulated genes in the cells transfected with miR-31-3p was predicted to be a target of hsa-miR-31-3p, and the only predicted one was the last target ranked.

The 25 putative direct target genes and the 27 indirect target genes were validated on qRT-PCR, out of these 47 genes, 45 displayed an expression level comparable to the level obtained in the array.

Finally, expression of these genes was analyzed in patient FFPE tumor samples and 2 of them showed a significant negative correlation with hsa-miR-31-3p expression levels: DBNDD2 and EPB41L4B (see FIGS. 1A and 1B).

In addition, using non-parametric differential analysis, these 2 genes were found to be associated to the progression free survival (p=0.004, for DBNDD2 and p=0.025 for EPB41L4B). Together, these results suggest that expression of DBNDD2 and EPB41L4B could distinguish between mCRC patients with poor or good prognosis, i.e. between non-responders and responders mCRC patients.

### Example 2

### Creation of a Tool with DBNDD2 and EPB41L4B Expression to Predict Response to EGFR Inhibitors

### Patients and Methods

### Patients

The set of patients was made of 20 mCRC patients, 13 males and 7 females. The median of age was 67±11.2 years. All had a metastatic disease at the time of the inclusion. All these patients developed a KRAS wild type metastatic colon cancer. All patients were considered refractory to a 5-fluorouracil-based regimen combined with irinotecan and oxaliplatin. They received an anti-EGFR-based chemotherapy, 8 patients with panitumumab, 10 patients with cetuximab and 2 patients received a combination of panitumumab and cetuximab. The number of chemotherapy lines before the introduction of Cetuximab and panitumumab was recorded. The median of follow-up until progression was 21 weeks and the median overall survival was 8.9 months.

### Measurement of Gene Expression

qRT-PCR of DBNDD2 and EPB41L4B expression on FFPE patients samples were performed on 20 ng of total RNA using ABI7900HT Real-Time PCR System (Applied Biosystem). All reactions were performed in triplicate. Expression levels were normalized to the GAPDH levels through the ΔΔCt method.

### Statistical Analyses

Survival statistical analysis was performed using the R packages ‘survival’ and ‘rms’. Univariate and multivariate analyses used a Cox proportional regression hazard model and generated a hazard ratio (HR). Nomograms were developed based on Cox proportional regression hazard models, which predict the probability of free-progression survival.

Gene and miRNA expression value comparison analyses were done using non-parametric test (Kruskal-Wallis tests) with the pairwise Wilcox test function in R.

The cor.test function was used to calculate Pearson correlations between expression values together with matching p-values. Statistical significance was set at p<0.05 for all analyses.

### Results

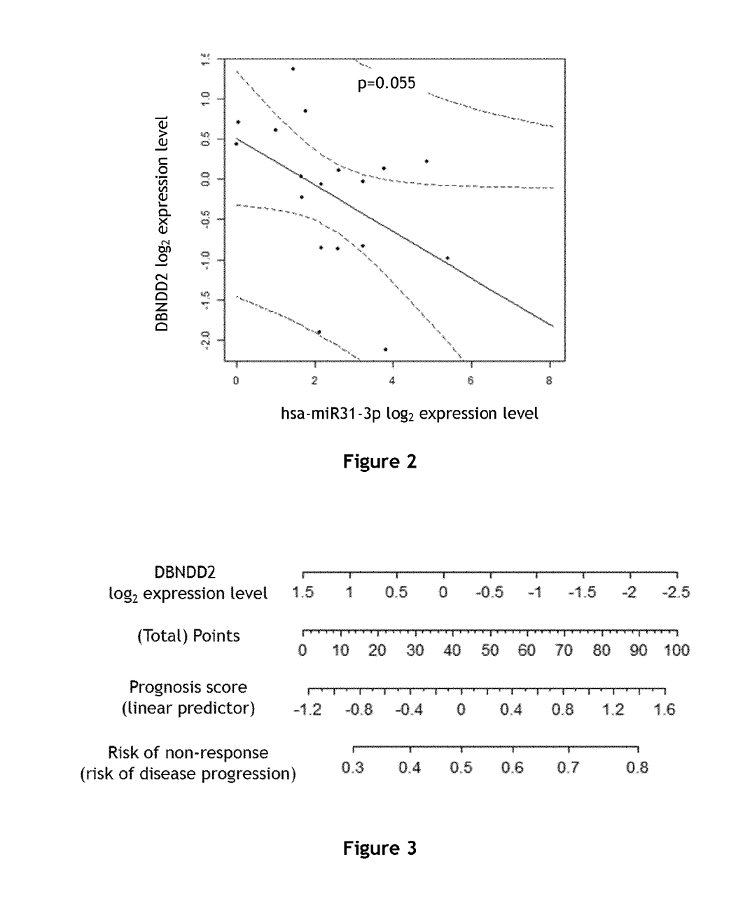

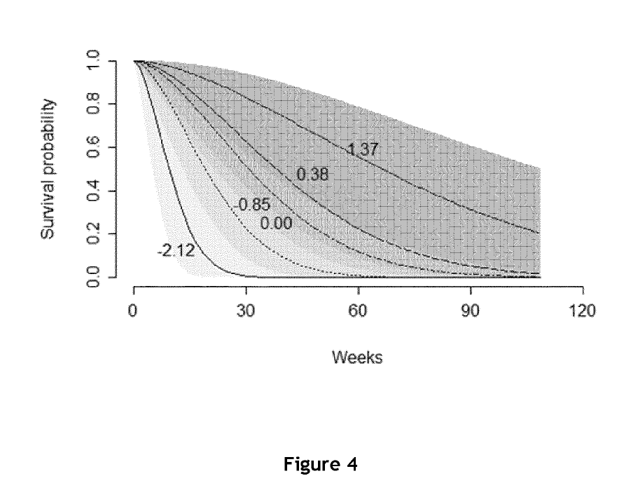

Expression of DBNDD2 and EPB41L4B was analyzed in the tumor samples. Statistical analyses showed a significant negative correlation with hsa-miR-31-3p expression levels: (see FIG. 2 for DBNDD2). In addition, using non-parametric differential analysis, these 2 genes were found to be associated to the progression free survival (p=0.025, for DBNDD2). Based on this results, to obtain a tool for predicting response of mCRC patient treated with anti-EGFR, multivariate Cox proportional hazards models status and log₂ of the gene expression as covariate were used to construct a nomogram based on PFS, thus permitting to predict the risk of progression (i.e. the risk of non-response, see FIGS. 3 and 4).

### Example 3

### Replication of the Predictive Value of DBNDD2 and EPB41L4B to EGFR Inhibitors in a New and Independent Cohort

### Patients and Methods

### Patients

The set of patients was made of 42 mCRC (metastatic colorectal cancer) patients, 27 males and 15 females. The median of age was 59±12.1 years. All had a metastatic disease at the time of the inclusion. All patients were treated with 3rd line therapy by a combination of irinotecan and panitumumab after progression with oxaliplatin and irinotecan chemotherapy based regimens. The median of follow-up until progression was 23 weeks and the median overall survival was 9.6 months. 26 samples were available in FFPE and 16 in frozen tissue.

### Measurement of Gene Expression

qRT-PCR validation of the target expression on frozen or FFPE patients samples were performed on 20 ng of total RNA using ABI7900HT Real-Time PCR System (Applied Biosystem). All reactions were performed in triplicate. Expression levels were normalized to the RNA18S or GAPDH levels through the ΔΔCt method.

### Statistical Analyses

Survival statistical analysis was performed using the R packages ‘survival’ and ‘rms’. Univariate and multivariate analyses used a Cox proportional regression hazard model. Gene and miRNA expression value comparison analyses were done using non-parametric test (Kruskal-Wallis tests) with the pairwise Wilcox test function in R.

Statistical significance was set at p<0.05 for all analyses.

### Results

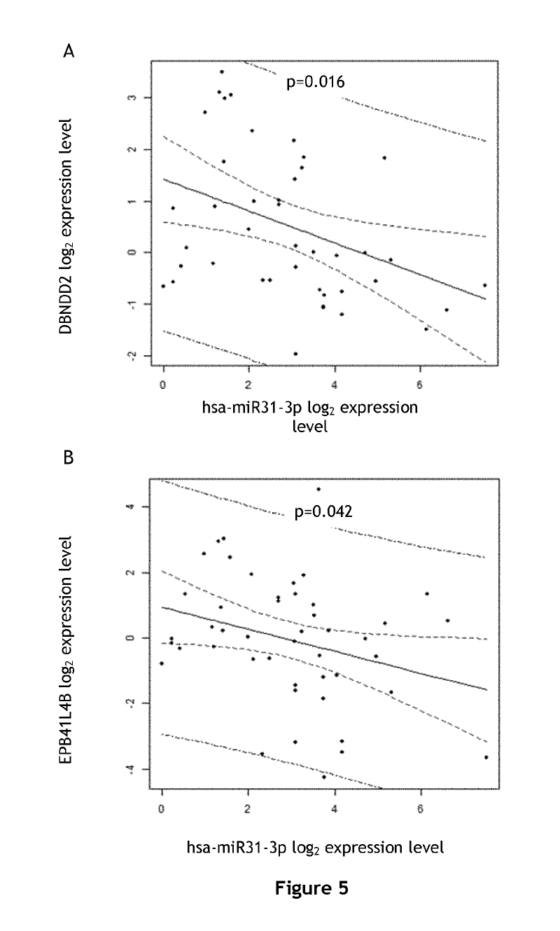

Expression of DBNDD2 and EPB41L4B was analyzed in the patient tumor FFPE samples. They showed a significant negative correlation with hsa-miR-31-3p expression levels: (see FIGS. 5A and 5B). A correlation between the expression of these two genes and prediction of response/non-response calculated based on the expression level of hsa-miR-31-3p as described in patent application PCT/EP2012/073535 was found (see FIG. 6).

Using a cox model, these 2 genes were found to be associated to the progression free survival (p=0.004 for DBNDD2 with GAPDH normalization and p=0.027 for EPB41L4B with RNA 18S normalization).

These results confirm that expression of DBNDD2 and EPB41L4B could discriminate mCRC patients with poor or good prognosis, i.e. between non-responders and responders mCRC patients.

### BIBLIOGRAPHIC REFERENCES

Albitar L et al. Mot Cancer 2010; 9:166;
Ambros V et al, RNA 2003 9(3):277-279;
Bair E. [symbol] R Tibshirani, PLOS Biology 2:511-522, 2004;
Bos. Cancer Res 1989; 49:4682-4689;
Bustin et al., 2005, Clin. Sci., 109:365-379;
Chan S L et al. Expert Opin Ther Targets. 2012 March; 16 Suppl 1:S63-8;
Chang K W et al. Oral Oncot. 2012 Jul. 30,
Chu H et al. Mutagenesis. 2012 Oct. 15;
Ciardello F et al. N Engl J Med. 2008 Mar. 13; 358(11):1160-74;
Cox, D. R. (1972). Journal of the Royal Statistical Society, Series B 34 (2), 187-220;
Cunningham et al, N Engl Med 2004; 351: 337-45;
Demiralay et al. Surgical Science, 2012, 3, 111-115;
Edkins et al. Cancer BiolTher. 2006 August; 5(8): 928-932
Eisenhauer et al, European Journal of Cancer, 2009, 45:228-247;
Griffiths-Jones S. NAR 2004 32(Database Issue):D109-D111;
Griffiths-Jones S et al. NAR 2006 34(Database Issue):D140-D144;
Griffiths-Jones S et al. NAR 2008 36(Database Issue):D154-D158;
Hatakeyama H. et al. PLoS One. 2010 Sep. 13; 5(9):e12702;
Kozomara A et al. NAR 2011 39(Database Issue):D152-D157;
Laurent-Puig P, et al, J Clin Oncol. 2009, 27(35):5924-30;
Leboulleux S et al. Lancet Oncol. 2012 September; 13(9):897-905;
Leslie K K et al. Gynecol Oncol. 2012 November; 127(2):345-50;
Li Y et al. Oncol Rep. 2010 October; 24(4):1019-28;
Liebner D A et al. Ther Adv Endocrinol Metab. 2011 October; 2(5):173-95;
Lievre et al, Cancer Res. 2006 66(8):3992-5;
Lièvre et al. J Clin Oncol. 2008 Jan. 20; 26(3):374-9;
Mimeault M et al. PLoS One. 2012; 7(2):e31919;
Mosakhani N. et al. Cancer Genet. 2012 October 22.doi:pii: S2210-7762(12)00229-3. 10.1016/j.cancergen.2012.08.003;
Ogino S, et al. J Mot Diagn 2008; 7:413-21;
Pan J et al. Head Neck. 2012 Sep. 13;
Ragusa M. et al. Mot Cancer Ther. 2010 December; 9(12):3396-409;
Schulz W A, et al. BMC Cancer. 2010 Sep. 22; 10:505;
Shepherd F A, et al, N Engl J Med 2005; 353:123-132;
Tam et al. Clin Cancer Res 2006; 12:1647-1653;
Thomasson M et al. Br J Cancer 2003, 89:1285-1289;
Thomasson M et al. 2012 May 3; 5:216;
U.S. Pat. No. 7,101,663;
Wang J, et al. Prostate. 2006 Nov. 1; 66(15):1641-52;
Wheeler D L et al. Nat Rev Clin Oncol. 2010 September; 7(9): 493-507;
WO2009/080437;
WO2010/121238;
WO2011/135459;
WO2010065940;
WO2010059742;
WO2009131710;
WO2007112097;
WO2011017106;
WO2010127338;
WO2007072225;
WO2008138578;
Xiao W et al. 2012. PLoS ONE 7(6): e38648;
Yin H et al. Biochemistry. 2006 Apr. 25; 45(16):5297-308;
Yu H et al. Mot Cancer Res 2010; 8:1501-1512;
Zeineldin R et al. J Oncol. 2010; 2010:414676,
Zhao L. et al. Int J Biochem Cell Biol. 2012 November; 44(11):2051-9.

1. An in vitro method for predicting whether a patient with a cancer is likely to respond to an epidermal growth factor receptor (EGFR) inhibitor, which method comprises determining the expression level of at least one target gene of hsa-miR-31-3p (SEQ ID NO:1) miRNA in a tumor sample of said patient, wherein said target gene of hsa-miR-31-3p is selected from DBNDD2 and EPB41 L4B.

2. The method of claim 1, wherein the patient has a KRAS wild-type cancer.

3. The method of claim 1, wherein the patient is afflicted with a cancer selected from colorectal, lung, breast, ovarian, endometrial, thyroid, nasopharynx, prostate, head and neck, liver, kidney, pancreas, bladder, and brain.

4. The method of claim 3, wherein the cancer is a colorectal cancer, in particular a metastatic colorectal cancer.

5. The method of claim 1, wherein the EGFR inhibitor is an anti-EGFR antibody, in particular cetuximab or panitumumab.

6. The method of claim 1, wherein the sample is a tumor tissue biopsy or whole or part of a tumor surgical resection.

7. The method of claim 1, wherein the level of expression of said at least one target gene of hsa-miR-31-3p is determined at the nucleic acid level by measuring in vitro the amount of transcripts produced by said target gene(s) of hsa-miR-31-3p, preferably by quantitative RT-PCR.

8. The method of claim 1, wherein the higher the level of expression of said at least one target gene of hsa-miR-31-3p is, the more likely the patient is to respond to the EGFR inhibitor treatment.

9. The method of claim 1, further comprising determining a prognostic score based on the expression level of said at least one target gene of hsa-miR-31-3p, wherein the prognostic score indicates whether the patient is likely to respond to the EGFR inhibitor.

10. The method of claim 1, wherein the prognostic score is of formula:
Prognosis score=a*x+b,
wherein:
x is the logged expression level of DBNDD2 measured in the patient's sample,
a and b are parameters that have been previously determined based on a pool of reference samples, and
the patient is predicted as responding or non-responding to the EGFR inhibitor if his/her prognosis score is greater or lower than a threshold value c, wherein the value of c has been determined based on the same pool of reference samples:
If a is positive, then the patient is predicted as responding to the EGFR inhibitor if his/her prognosis score is greater than or equal to threshold value c, and not responding to the EGFR inhibitor if its prognosis score is lower than threshold value c,
If a is negative, then the patient may be predicted as responding to the EGFR inhibitor if his/her prognosis score is lower than or equal to threshold value c, and not responding to the EGFR inhibitor if his/her prognosis score is greater than threshold value c.

11. The method of claim 1, wherein the prognostic score is of formula:
Prognosis score=a*x+b,
wherein:
x is the logged expression level of DBNDD2 measured in the patient's sample,
a and b are parameters that have been previously determined based on a pool of reference samples, and
depending if a is positive or negative:
If a is positive, the higher the prognosis score, the higher is the probability of response to the EGFR inhibitor treatment;
if a is negative, then the lower the prognosis score, the higher is the probability of response to the EGFR inhibitor treatment.

12. The method of claim 1, further comprising determining a risk of non-response based on a nomogram calibrated based on a pool of reference samples.

13. The method of claim 1, further comprising determining at least one other parameter positively or negatively correlated to response to EGFR inhibitors, and calculating a composite score taking into account the expression level of said at least one target gene of hsa-miR-31-3p and said other parameter(s), wherein the composite score indicates whether the patient is likely to respond to the EGFR inhibitor.

14. A kit for determining whether a patient with a cancer is likely to respond to an epidermal growth factor receptor (EGFR) inhibitor, comprising or consisting of:
a) reagents for determining the expression level of at least one target gene of hsa-miR-31-3p (SEQ ID NO:1) miRNA in a sample of said patient, wherein said target gene of hsa-miR-31-3p is selected from DBNDD2 and EPB41 L4B, and
b) reagents for determining at least one other parameter positively or negatively correlated to response to EGFR inhibitors, wherein said reagents are selected from:
i) reagents for determining the expression level of at least one miRNA positively or negatively correlated to response to EGFR inhibitors, in particular hsa-miR-31-3p (SEQ ID NO:1) miRNA or hsa-miR-31-5p (SEQ ID NO:34) miRNA, and/or
ii) reagents for detecting at least one mutation positively or negatively correlated to response to EGFR inhibitors.

15. An EGFR inhibitor for use in treating a patient affected with a cancer, wherein the patient has been classified as being likely to respond to the EGFR inhibitor by the method according to claim 1.

16. An EGFR inhibitor for use in treating a patient affected with a cancer, wherein said treatment comprises a preliminary step of predicting if said patient is or not likely to respond to the EGFR inhibitor by the method according to claim 1, and said EGFR inhibitor is administered to the patient only is said patient has been predicted as likely to respond to the EGFR inhibitor by the method according to any one of claims 1 to 13.

17. A method for treating a patient affected with a cancer, which method comprises:
(i) determining whether the patient is likely to respond to an EGFR inhibitor, by the method according to the invention, and
(ii) administering an EGFR inhibitor to said patient if the patient has been determined to be likely to respond to the EGFR inhibitor.

18. The method according to claim 17, further comprising, if the patient has been determined to be unlikely to respond to the EGFR inhibitor, a step (iii) of administering an alternative anticancer treatment to the patient.

19. The method according to claim 18, wherein said alternative anticancer treatment is selected from:
a) a VEGF inhibitor,
b) a VEGF inhibitor in combination with FOLFOX,
c) a VEGF inhibitor in combination with FOLFIRI,
d) 5-FU, and
e) 5-FU in combination with Mitomycin B.


SEQUENCE LISTING (33 sequences)

SEQ ID NO: 1 - RNA, 22 nt, homo sapiens
ugcuaugccaacauauugccau

SEQ ID NO: 2 - DNA, 1165 bp, homo sapiens
gtgcttggtaggttgggcggcggctctgtctagtcccagagccaggaatcaggggcagccgggcgagtcccagggcaggggtcctccgccgccttgcacctgccctgctgggcggcaccgggtcagtgccctgccccctcctgcgggtcccaactctctctttcccatcgtgcgtcctctggagaagtgcgcgcgtgagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 3 - DNA, 1114 bp, homo sapiens
ctcgttcccggctcccagggcccgtcggtcccccgggagccctggaggcgcagccccaccccggccggcgcggctcgctcccacgcccccgccgcggcctcgctggagcggacggactgagtcagagggggcgccagcgctgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 4 - DNA, 998 bp, homo sapiens
cccggcgcccaccccgcccagtgtgcttggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 5 - DNA, 1044 bp, homo sapiens
atcgtgcgtcctctggagaagtgcgcgcgtgtaagtgtggcgagtgtggccaagggtgccggaggcagggttcgggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 6 - DNA, 1263 bp, homo sapiens
gtgcttggtaggttgggcggcggctctgtctagtcccagagccaggaatcaggggcagccgggcgagtcccagggcaggggtcctccgccgccttgcacctgccctgctgggcggcaccgggtcagtgccctgccccctcctgcgggtcccaactctctctttcccatcgtgcgtcctctggagaagtgcgcgcgtgagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctggtatgcccctctgctttggggacttcagtgccagtcagccagagccggatgtcaggctctgaaacgaggctacaaggctgggctggggaagtacacaaggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 7 - DNA, 1212 bp, homo sapiens
ctcgttcccggctcccagggcccgtcggtcccccgggagccctggaggcgcagccccaccccggccggcgcggctcgctcccacgcccccgccgcggcctcgctggagcggacggactgagtcagagggggcgccagcgctgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctggtatgcccctctgctttggggacttcagtgccagtcagccagagccggatgtcaggctctgaaacgaggctacaaggctgggctggggaagtacacaaggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 8 - DNA, 1440 bp, homo sapiens
tgggaaggggtgggctgcagcactggaggaagggaaccctccaccctgagatctctgtctctatcctatcctgtccctggccttctgaggcaagcggggccaattaaggggaaaacgtacctcctccatttgtgctgaaccaatccctccaacccctctcaggagggcatgatatggagagttgggcattggctgtgttccctgaatacagagtatctctcttgtggtgcctggaactggcatcccctttgtggagcttagggcaagccccgcctctgcatgagacttggtttgtgggacacacttggtttcagggaaggggaaagaggtcaccaagggcagaggtgtccaggccggagccaggggccccactgttgggatgctggctgcagtggggcgccccaagcccaggtcccctctgtcttctctttcgactttgcagctgtacttgttttgctcctctacccgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 9 - DNA, 1538 bp, homo sapiens
tgggaaggggtgggctgcagcactggaggaagggaaccctccaccctgagatctctgtctctatcctatcctgtccctggccttctgaggcaagcggggccaattaaggggaaaacgtacctcctccatttgtgctgaaccaatccctccaacccctctcaggagggcatgatatggagagttgggcattggctgtgttccctgaatacagagtatctctcttgtggtgcctggaactggcatcccctttgtggagcttagggcaagccccgcctctgcatgagacttggtttgtgggacacacttggtttcagggaaggggaaagaggtcaccaagggcagaggtgtccaggccggagccaggggccccactgttgggatgctggctgcagtggggcgccccaagcccaggtcccctctgtcttctctttcgactttgcagctgtacttgttttgctcctctacccgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctggtatgcccctctgctttggggacttcagtgccagtcagccagagccggatgtcaggctctgaaacgaggctacaaggctgggctggggaagtacacaaggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 10 - DNA, 1486 bp, homo sapiens
cgagtgtggccaagggtgccggaggcagggttcgggtgcgtagtcgttgcgtgggcgctgcccaaaaggcgcagagcatcaagtgtgcgtgggcagaaccggcgcgggcgcccgccgcgggtctgcgcggggcgggggcgcagcaagtgcatccgagcgagcggagactagcgcaccggcgtcggtggcgagggtggtgcagaggagtccggctgggcggagggaggaaggatgggtgcgggtaactttttgaccgccttggaagtaccagtagccgcgctcgcaggggctgcctccgaccgccgggcgagctgcgagcgagtgagcccgccaccgcccctcccccacttccgcctcccgcctcttcctcgttcccggctcccagggcccgtgtccaggccggagccaggggccccactgttgggatgctggctgcagtggggcgccccaagcccaggtcccctctgtcttctctttcgactttgcagctgtacttgttttgctcctctacccgcaggagctgacatggacccaaatcctcgggccgccctggagcgccagcagctccgccttcgggagcggcaaaaattcttcgaggacattttacagccagagacagagtttgtctttcctctgtcccatctgcatctcgagtcgcagagaccccccataggtagtatctcatccatggaagtgaatgtggacacactggagcaagtagaacttattgaccttggggacccggatgcagcagatgtgttcttgccttgcgaagatcctccaccaaccccccagtcgtctgggatggacaaccatttggaggagctgagcctgccggtgcctacatcagacaggaccacatctaggacctcctcctcctcctcctccgactcctccaccaacctgcatagcccaaatccaagtgatgatggagcagatacgcccttggcacagtcggatgaagaggaggaaaggggtgatggaggggcagagcctggagcctgcagctagcagtgggcccctgcctacagactgaccacgctggctattctccacatgagaccacaggcccagccagagcctgtcgggagaagaccagactctttacttgcagtaggcaccagaggtgggaaggatggtgggattgtgtacctttctaagaattaaccctctcctgctttactgctaattttttcctgctgcaaccctcccaccagtttttggcttactcctgagatatgatttgcaaatgaggagagagaagatgaggttggacaagatgccactgcttttcttagcactcttccctcccctaaaccatcccgtagtcttctaatacagtctctcagacaagtgtctctagatggatgtgaactccttaactcatcaagtaaggtggtactcaagccatgctgcctccttacatcctttttggaacagagcacggtataaataataaactaataataatatgccaaccaaaaaa

SEQ ID NO: 11 - protein, 161 aa, homo sapiens
MDPNPRAALERQQLRLRERQKFFEDILQPETEFVFPLSHLHLESQRPPIGSISSMEVNVDTLEQVELIDLGDPDAADVFLPCEDPPPTPQSSGMDNHLEELSLPVPTSDRTTSRTSSSSSSDSSTNLHSPNPSDDGADTPLAQSDEEEERGDGGAEPGACS

SEQ ID NO: 12 - protein, 161 aa, homo sapiens; identical to SEQ ID NO: 11

SEQ ID NO: 13 - protein, 161 aa, homo sapiens; identical to SEQ ID NO: 11

SEQ ID NO: 14 - protein, 161 aa, homo sapiens; identical to SEQ ID NO: 11

SEQ ID NO: 15 - protein, 112 aa, homo sapiens
MDPNPRAALERQQLRLRERQKFFEDILQPETEFVFPLSHLHLESQRPPIGSISSMEVNVDTLEQVELIDLGDPDAADVFLPCEDPPPTPQSSGMPLCFGDFSASQPEPDVRL

SEQ ID NO: 16 - protein, 112 aa, homo sapiens; identical to SEQ ID NO: 15

SEQ ID NO: 17 - protein, 263 aa, homo sapiens
MESWALAVFPEYRVSLLWCLELASPLWSLGQAPPLHETWFVGHTWFQGRGKRSPRAEVSRPEPGAPLLGCWLQWGAPSPGPLCLLFRLCSCTCFAPLPAGADMDPNPRAALERQQLRLRERQKFFEDILQPETEFVFPLSHLHLESQRPPIGSISSMEVNVDTLEQVELIDLGDPDAADVFLPCEDPPPTPQSSGMDNHLEELSLPVPTSDRTTSRTSSSSSSDSSTNLHSPNPSDDGADTPLAQSDEEEERGDGGAEPGACS

SEQ ID NO: 18 - protein, 214 aa, homo sapiens
MESWALAVFPEYRVSLLWCLELASPLWSLGQAPPLHETWFVGHTWFQGRGKRSPRAEVSRPEPGAPLLGCWLQWGAPSPGPLCLLFRLCSCTCFAPLPAGADMDPNPRAALERQQLRLRERQKFFEDILQPETEFVFPLSHLHLESQRPPIGSISSMEVNVDTLEQVELIDLGDPDAADVFLPCEDPPPTPQSSGMPLCFGDFSASQPEPDVRL

SEQ ID NO: 19 - protein, 259 aa, homo sapiens
MGAGNFLTALEVPVAALAGAASDRRASCERVSPPPPLPHFRLPPLPRSRLPGPVSRPEPGAPLLGCWLQWGAPSPGPLCLLFRLCSCTCFAPLPAGADMDPNPRAALERQQLRLRERQKFFEDILQPETEFVFPLSHLHLESQRPPIGSISSMEVNVDTLEQVELIDLGDPDAADVFLPCEDPPPTPQSSGMDNHLEELSLPVPTSDRTTSRTSSSSSSDSSTNLHSPNPSDDGADTPLAQSDEEEERGDGGAEPGACS

SEQ ID NO: 20 - DNA, 3783 bp, homo sapiens
caggccgccggcccgggagagcgcgcggcggcggcggcggcgggacggccccgagcgcgccctcccgcctgcggccactcgcagccggcgctggccgggccggcgcgccccgcaggcggctagagcgcggcctcggcctcggcgcgggcttgccccgggccgtagcccgcgagaggcggcgcgggcggccgagggcactgacggcgtcgcgggacgctcccgggcggcggcgcagcggcagcggcagcggcagcggcagcgcagggggcggaggcagggggcgcccccagccaggatgctgcggttcctgcgccggacctttggccgccgctccatgcagcgctacgcgcggggcgcggcggggcgcggggccgccgggctgggggacgagcgcgatggggggccacgggggggcccggccgccgccgcctcctcctcggcgctgcccgccgcgcccgggggcagcgtgttcccggcgggcggcgggcccctgctcaccggcggcgcggccgtgcacatctccgccgccggcgccgccaaggccaccctctactgccgcgtcttcctgctcgacgggaccgaagtgagcgtggacctgccgaaacatgccaaaggccaggatttgtttgatcagattgtgtaccacttggaccttgtggaaacagattactttggcctccagttcctcgactctgcccaggttgcgcactggctggatcatgccaaacccataaaaaagcagatgaaaattggacctgcttatgctttacactttcgagttaaatactattcttcagaaccaaacaaccttcgtgaggagtttacaaggtacctgtttgttttacaactcaggcatgacattctttctggaaaattgaaatgcccttatgaaacagctgtggaattagctgctctctgtctacaagcggagcttggggagtgcgagcttccagaacacacaccagagcttgtgtctgagtttcggttcattccaaatcagacagaagcaatggaatttgatatcttccagagatggaaagagtgcaggggaaagagccctgcccaggcggaactctcctatctgaataaagcgaagtggctggaaatgtatggggtagacatgcacgttgtcaggggaagagatggctgtgaatattctcttggactgaccccgacaggcatattaatctttgaaggagctaacaaaataggcttattcttttggcctaaaattaccaaaatggattttaaaaagagcaaattgacactcgtggtggtcgaggatgatgatcagggacgtgagcaagagcacacgtttgtgttccggttagacagtgccaggacctgcaaacacctttggaagtgtgcagttgagcaccacgcattcttccgactgcggacgccaggaaacagcaaatccaatagatccgactttatcaggctgggctctcgcttcagattcagtgggcggacagaatatcaagctacacatggctccaggttacgaagaaccagcacctttgagaggaagcctagtaaacgttatccatcccggagacattcaacgttcaaagcaagcaacccagtgatagcagcccagctctgctctaaaacaaatccagaagtccataattaccagcctcaatatcatcctaatatccatcccagccagccccggtggcatcctcactctccaaatgtcaggccatcctttcaggatgacaggtcgcattggaaagcatcggccagtggagatgacagccattttgattatgtccacgaccagaaccagaagaacttaggagggatgcaaagtatgatgtatcgagataaactcatgactgcactttgagagactgaagcatctctcttccattcaccttcatagtttcattgcattccatgaaaagtgtcttggcctcagatggatggatgtgtttggacgagtgtctttaaggagtagtcctgaaaggtgtttttggtgtccatgtaaatatttgaagataaaaccactatagcttgtcataatttactgttgactgcattctcattaaaatgaaggtaaaggctcaggaatcatattgatgttctgattttaaaattggagtcaaagtctatgtttatcattttactatgttcctgatgttctttgttatttaattaatgggagcaaataaaaccagaagagcttgggaagattgctcagcatatattcctgtcgtagaagttgagattgctagggtccagtttccctagtgtggcctggacgagtcatttccccttcattgacctcattttccccatctgaaaagagagggttggactaagtgatctccaaggtcctttccaactctaaaattctgcaatttgttaacatttcattttgtttaggttgaggacatacattcaaactaattttatcacaaggaaaactgcaatacccacttccttgacagagttactcctttcagaagctaaataaagtatataacttattagatgttatatagatacagggggactttgaatttcacatcttaaagcagttgagctactttgaatttaagcagtcgtactaatcttaaattgcatagcatttgttttgatcgaatttgctgctcaagtatgggaataatttttaatgtcttaatgattggtgctgctaacttgcgtgatttcagaagacataattgtgaatacacactgtcagaattgggggattggtttttaccctagacttcactcttaaaaagcaacgtgcaatcaagatcatttatggctcaaatgaaagcatataaggttttcttgaagttgtgccaaagcattctgtagagtaggatgagatggttgttgccctagtctgttggtagaaccagaaatcaatatgttgtcttttaggttaaagcttgtaccaaaatatttatttcccccatttcaagccctgagtcaaacatttttttctcttaataatagacctgaaatgttttattagtatttctgtgaaatcagttgattcttgtgccatttttgtatatgtaattgtaattttgcccatgttaggccctctaaaaaatgtttgacatcctttgagatattttattactaaaatctgatcttttttggctactgcaaaaatctattcagcaagaaggtatcagctgcataccttgcacagtggagctgactacctataaactctccctaaggcatttgtttacaggtgtattccattttagcagacgttctgatgctcagtgtatgtgctgcatacaaataaatgtgttctgaatcttttcatcttattgatagcatttttacaaatgtgtttccaaggaataaagattattcttgctttttttttgactccatcttcattttttttaaattgattcttgttgctatgcagaagtctcatttgtgaatgaccttggtaacagaacagttggcttttggaagtctgaaggtgagcattcagttaggtgggtggagcaagatcatcctagaatgaggctgctcttggcaagagtggatcttataggcacagcagctgatgcctttcttcatctggggcaactctggtgaaggttgtcctgcctgtcacaggtgctgagtagagagaagtggtggcagtgggatttcctcagtaatagtcctgtaaaggtacgtgtttgtcctggctacttgtgctcttcctggcaggaaggcatccaaacccttatctgtgggctcctggaaattgtgtatgccatataataccctctaataaatacctctctgcttaaa

SEQ ID NO: 21 - DNA, 5578 bp, homo sapiens
caggccgccggcccgggagagcgcgcggcggcggcggcggcgggacggccccgagcgcgccctcccgcctgcggccactcgcagccggcgctggccgggccggcgcgccccgcaggcggctagagcgcggcctcggcctcggcgcgggcttgccccgggccgtagcccgcgagaggcggcgcgggcggccgagggcactgacggcgtcgcgggacgctcccgggcggcggcgcagcggcagcggcagcggcagcggcagcgcagggggcggaggcagggggcgcccccagccaggatgctgcggttcctgcgccggacctttggccgccgctccatgcagcgctacgcgcggggcgcggcggggcgcggggccgccgggctgggggacgagcgcgatggggggccacgggggggcccggccgccgccgcctcctcctcggcgctgcccgccgcgcccgggggcagcgtgttcccggcgggcggcgggcccctgctcaccggcggcgcggccgtgcacatctccgccgccggcgccgccaaggccaccctctactgccgcgtcttcctgctcgacgggaccgaagtgagcgtggacctgccgaaacatgccaaaggccaggatttgtttgatcagattgtgtaccacttggaccttgtggaaacagattactttggcctccagttcctcgactctgcccaggttgcgcactggctggatcatgccaaacccataaaaaagcagatgaaaattggacctgcttatgctttacactttcgagttaaatactattcttcagaaccaaacaaccttcgtgaggagtttacaaggtacctgtttgttttacaactcaggcatgacattctttctggaaaattgaaatgcccttatgaaacagctgtggaattagctgctctctgtctacaagcggagcttggggagtgcgagcttccagaacacacaccagagcttgtgtctgagtttcggttcattccaaatcagacagaagcaatggaatttgatatcttccagagatggaaagagtgcaggggaaagagccctgcccaggcggaactctcctatctgaataaagcgaagtggctggaaatgtatggggtagacatgcacgttgtcaggggaagagatggctgtgaatattctcttggactgaccccgacaggcatattaatctttgaaggagctaacaaaataggcttattcttttggcctaaaattaccaaaatggattttaaaaagagcaaattgacactcgtggtggtcgaggatgatgatcagggacgtgagcaagagcacacgtttgtgttccggttagacagtgccaggacctgcaaacacctttggaagtgtgcagttgagcaccacgcattcttccgactgcggacgccaggaaacagcaaatccaatagatccgactttatcaggctgggctctcgcttcagattcagtgggcggacagaatatcaagctacacatggctccaggttacgaagaaccagcacctttgagaggaagcctagtaaacgttatccatcccggagacattcaacgttcaaagcaagcaacccagtgatagcagcccagctctgctctaaaacaaatccagaagtccataattaccagcctcaatatcatcctaatatccatcccagccagccccggtggcatcctcactctccaaatgtcagctacccgctcccttccccagtgcttagcagctcggaccggttgccttttggcattgaggagaatgggggcacaccgttcctcaccgcagcttcaggaaggcatcaccaccagcaccagcatcagcatcagcaccagcaccactcaaactacagcctctcactgaccctggagaacaaagaggggcctctgaggtccccaaactccagcagcaagtcccttacaaaactgagtccaggaacacctgccttgttcagtgaagccgctgcccatctaaagaagctggaactggaaactgtgaaggctgctggaccctggcctcctctgcacatcaacataaacaaggctgaagaaaagaaagtctcggagaaaactcttcagactccacttttgccttcccctgttgcggatcatgtgaagtgtaacattctgaaagcccagttggaaaatgcttcccgagtgaacatccagggtggaaaggaggaatcaccgtttgtaaatatcaataagaaatccagtcttcaggacgctagtgtaagaagtcctattcctattcgtgtggaaactgcccagccagctgtggaaaagccggaaatcaagcctccccgagtgaggaagttaacaagacagtatagttttgatgaagacgacctccctccagacctggccgaggcagtgggagtgaccacatctacaaccacaaacaccacaacggccgccacacaagtctccgtgccgctgccgtcccccaaggtccagaatgtcagctcgcctcacaagtcagaaggcaaaggcctgctgtcccctggggccaagagcccctctgaccgaggaggtgcctttaccctggagccgggtgatcttctgatggatttcacagaagccactcctctggcagagcccgccagcaacccccactgtgcccactctcgctgttctcctccactctctctccccatgaaggaagagaccactggagtttgcatgtaccctccaatcaaaacgaggctgataaaaacattcccggttgatacaatgaacccgtttcctgatactttcaccacagggccacagtttactgcagacttcagagacagtaaattacagtgctgtcctggcccgacttccccgctgatcccagcagcgaccctgaggcctttgacagagaccgtctccacagtgcagaccatttacaccacccggaaacctgtttctctggcagccagtgcagagacactccggcaggaactggagagagagaagatgatgaaaagactgttgatgaccgaactgtgaaattctccccttgtcacctggaagatggcatggtgccttctgtccgtcttctttcttcgggctttgtgtgctcactctagcacagcatacaagtgtgtgctctgttcgcccaggtctccatggttagttgaagccaatttctggcttgacttttatgggaaaagttattttatgtctcctaagcattagagtttttctattactctatgtagttgagacaggatttgataagtctaggaaaagaaagatgggaaaacgggattccttttcagaagtacctgtgtgtatctgttaataaccacaggggttaatatgatgtaggatcttttactatcaatttcaaccatttgattttgtatgattgaaacttgcaccgagctttgactgtttgttaaagagtcatttttaatgaaagaataattctttattgctggtttttcatttacactgataaatacacagatcttataaagtctttaacattcatttgtattcagatgtgagtagaagaactaaaaaaagaaagttacatatcactatgactgaaggtacttcagcttaatctgaaatataatttaacttgtgaactccttggatatgatattatttggaataaacagaatttatcattgaacccaaagtaggaaatgatagcttacattgtctaaaaatccttacaaggttaagatgattcaatatcaagaagattcagaaaattatttctaaagttgatcgattcatgtcgtattgatagaatcttgaccagaagaaattttgctctttttatatagtttcaagaaatgtgtttttaaatttttattaatgcacttgaacaactttgcaggaataaagcaaccccctaaccacaaaatatccctctaaattagttccctagctttctcaatgaatacacacatatttttacatagctatgatcgttgtgtacattctcctttgttttacttctcggcctaacacttgtctcctcttgtcaacacagattctactctcaccaatttaaatgtctttatatccatgtaacatgggtaacctcacttcaccccattattagatatttgagttatatctaatttttcactcttataaatagtgctgctatgaatgtctgtaaaaaaaaaaaactgctccttcttttggattattcccttaggaatatctccaaagagggattacaaggtcaaagagcatgaagtattttatagctcttgttttatattgccagattgctttctagaaagatccaatctttgggttggaaggaccttaaaggtcatctagtttagcctccccaccccctctgaatgcttgaatcccctcgacaatttatgatgccaccagcaatgtataagcatttctgtttaccaatagctctgccagtattgggttttgccatttttatttatttttgctagtttaataggtatgtatagttgttcttgaagagttgttttatttcattaattgctagcaaggctgagcacttttccatgtgatgatttactagttgtatttccttgtgtgtaaaatgttcattcatttcttatgaccacttgttaagaggaactgatctcatatatttgtatcagaactgtatttttatgttatattgtatagtttgctctcctgcccctctccttaaaactgaatggtgccaataatttgatactaatgactacaaaaaaaggtaatgcctcatttactagtattgttgtaaaatgaggaatgtatgtgaatattcagataaccgaggattaaccctttaagtgctgaatctttaaaattttaatatatttttttttgagggaaatctttctaaaatgtattacgcacttccctgccttagtaaacagagtatactggagagtatttaaccttttcttgatgagtcatggtcatgattataaacatcagccccttttataccttggtacggtgcagtgatatcattaagagctatcaatatgtgtagggcttggcttggccttttataggatattatgctgttctcactgatggttttttactgctctctgctctgtcagtggagctatccggggcaattgtagcgtttgggtccttttacccctatgtcccccggctatacttttaaaacagctttagctgttctttatcttgtgcacatgatacaaaatatgttcccgtacaatatggggctgtcacttcttgccaacccagcaccctcttcctcttctaacctgctttctgaggcttctgctcttcacctcctgctcgctgatggaaacctccagggcaaagctgaaggtttcttggggaagccaggaaagccagtatttcctatgtgtcagatctgcttggcttccaagaagggatgcatgggctttttggccagtgtttccaggaggctctgggcttcctgcttcttccccgcttcccccagagttcacagatgttgaagtttctgaaggttgacgtcactggaagtctgaccacaaacaagttggctgttactgtatttgaaacccagtacctttggcagctcacctctaaccagtaaaataagaggattccatggtttca

SEQ ID NO: 22 - protein, 518 aa, homo sapiens
MLRFLRRTFGRRSMQRYARGAAGRGAAGLGDERDGGPRGGPAAAASSSALPAAPGGSVFPAGGGPLLTGGAAVHISAAGAAKATLYCRVFLLDGTEVSVDLPKHAKGQDLFDQIVYHLDLVETDYFGLQFLDSAQVAHWLDHAKPIKKQMKIGPAYALHFRVKYYSSEPNNLREEFTRYLFVLQLRHDILSGKLKCPYETAVELAALCLQAELGECELPEHTPELVSEFRFIPNQTEAMEFDIFQRWKECRGKSPAQAELSYLNKAKWLEMYGVDMHVVRGRDGCEYSLGLTPTGILIFEGANKIGLFFWPKITKMDFKKSKLTLVVVEDDDQGREQEHTFVFRLDSARTCKHLWKCAVEHHAFFRLRTPGNSKSNRSDFIRLGSRFRFSGRTEYQATHGSRLRRTSTFERKPSKRYPSRRHSTFKASNPVIAAQLCSKTNPEVHNYQPQYHPNIHPSQPRWHPHSPNVRPSFQDDRSHWKASASGDDSHFDYVHDQNQKNLGGMQSMMYRDKLMTAL

SEQ ID NO: 23 - protein, 900 aa, homo sapiens
MLRFLRRTFGRRSMQRYARGAAGRGAAGLGDERDGGPRGGPAAAASSSALPAAPGGSVFPAGGGPLLTGGAAVHISAAGAAKATLYCRVFLLDGTEVSVDLPKHAKGQDLFDQIVYHLDLVETDYFGLQFLDSAQVAHWLDHAKPIKKQMKIGPAYALHFRVKYYSSEPNNLREEFTRYLFVLQLRHDILSGKLKCPYETAVELAALCLQAELGECELPEHTPELVSEFRFIPNQTEAMEFDIFQRWKECRGKSPAQAELSYLNKAKWLEMYGVDMHVVRGRDGCEYSLGLTPTGILIFEGANKIGLFFWPKITKMDFKKSKLTLVVVEDDDQGREQEHTFVFRLDSARTCKHLWKCAVEHHAFFRLRTPGNSKSNRSDFIRLGSRFRFSGRTEYQATHGSRLRRTSTFERKPSKRYPSRRHSTFKASNPVIAAQLCSKTNPEVHNYQPQYHPNIHPSQPRWHPHSPNVSYPLPSPVLSSSDRLPFGIEENGGTPFLTAASGRHHHQHQHQHQHQHHSNYSLSLTLENKEGPLRSPNSSSKSLTKLSPGTPALFSEAAAHLKKLELETVKAAGPWPPLHININKAEEKKVSEKTLQTPLLPSPVADHVKCNILKAQLENASRVNIQGGKEESPFVNINKKSSLQDASVRSPIPIRVETAQPAVEKPEIKPPRVRKLTRQYSFDEDDLPPDLAEAVGVTTSTTTNTTTAATQVSVPLPSPKVQNVSSPHKSEGKGLLSPGAKSPSDRGGAFTLEPGDLLMDFTEATPLAEPASNPHCAHSRCSPPLSLPMKEETTGVCMYPPIKTRLIKTFPVDTMNPFPDTFTTGPQFTADFRDSKLQCCPGPTSPLIPAATLRPLTETVSTVQTIYTTRKPVSLAASAETLRQELEREKMMKRLLMTEL

SEQ ID NO: 24 - protein, 188 aa, homo sapiens [sequence omitted]

SEQ ID NO: 25 - protein, 766 aa, homo sapiens [sequence omitted]

SEQ ID NO: 26 - protein, 189 aa, homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 27 - protein, 1068 aa, homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 28 - protein, 1210 aa, homo sapiens [sequence omitted]

SEQ ID NO: 29 - protein, 705 aa, homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGPGNESLKAMLFCLFKLSSCNQSNDGSVSHQSGSPAAQESCLGWIPSLLPSEFQLGWGGCSHLHAWPSASVIITASSCH

SEQ ID NO: 30 - protein, 628 aa, homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGS

SEQ ID NO: 31 - protein, 480 aa, homo sapiens [sequence omitted]

SEQ ID NO: 32 - protein, 480 aa, homo sapiens [sequence omitted]

SEQ ID NO: 33 - protein, 480 aa, homo sapiens [sequence omitted]